(19) United States
(12) Patent Application Publication
(10) Pub. No.: US 2012/0270297 A1
(43) Pub. Date: Oct. 25, 2012

(54) CULTURING AND GENETIC MANIPULATIONS OF THERMOTOGA SPP.

(75) Inventors: Zhaohui Xu, Bowling Green, OH (US); Dongmei Han, Bowling Green, OH (US)

(73) Assignee: BOWLING GREEN STATE UNIVERSITY, Bowling Green, OH (US)

(21) Appl. No.: 13/432,907

(22) Filed: Mar. 28, 2012

Related U.S. Application Data
(60) Provisional application No. 61/468,279, filed on Mar. 28, 2011.

(51) Int. Cl. C12N 1/20; C07H 21/04; C12N 1/21; C12N 15/63; C12N 9/10; C12N 9/16

(52) U.S. Cl. 435/193; 435/196; 435/252.1; 435/252.3; 435/252.33; 435/320.1; 536/23.2

(57) ABSTRACT

Described herein is the creation and use of Thermotoga-E. coli shuttle vectors; an embedded cultivation method that greatly simplifies the cultivation methods for Thermotoga previously used; and the subcloning, characterization, and use of a Thermotoga Restriction-modification system.

### CROSS-REFERENCE TO RELATED APPLICATIONS

This application is claiming the benefit, under 35 U.S.C. §119 (e), of the provisional application filed Mar. 28, 2011 under 35 U.S.C. §111 (b), which was granted Ser. No. 61/468,279. This provisional application is hereby incorporated by reference in its entirety.

### BACKGROUND

Besides Aquifex, Thermotoga are the only group of bacteria that can grow up to 90° C. Isolates of Thermotoga have been discovered from heated sea floors, continental hot springs, and oil fields. Analysis of their 16S rRNA sequences have positioned Thermotoga spp. to a deep branch of the tree of life, suggesting that these strict anaerobes emerged at an early stage of evolution, when the surface of the Earth was hot and its atmosphere contained little oxygen. Study of the molecular genetics of Thermotoga is expected to shed light on the fundamental questions related to the origin of life as well as the mechanisms of the thermostability of macromolecules under extreme conditions. Many Thermotoga enzymes have been expressed in E. coli and display extraordinary stability and extended shelf life. Also, importantly, Thermotoga hydrolyze a number of polysaccharides, including cellulose, through fermentative catabolism and produce hydrogen gas as one of the final products, as well as materials for bioplastics. These properties have stimulated tremendous interest in utilizing these bacteria for industrial purposes, such as utilizing these bacteria to produce biomass-based clean energy, especially through metabolic engineering approaches. However, due to the lack of simple cultivation methods and genetic tools, the investigations of Thermotoga are still largely limited to biochemical, genomic, and fermentative studies, as is the case with most hyperthermophiles

This disclosure presents advances in culturing and genetic manipulations of Thermotoga spp.

I. Method for Preparation and Handling Solid Cultures of Thermotoga Spp. Under Aerobic Conditions:

The success of obtaining isolated single colonies from solid media is essential to any genetic manipulation with microbes, because each single colony represents one pour strain and all the cells forming that colony share the same genetic information. The cultivation of these bacteria is challenging because they grow best around 80° C. and they do not grow if oxygen is present.

Traditionally they are handled in an anaerobic glove box, which is expensive and cumbersome to use. In lieu of an anaerobic chamber, one may use a stream of high pressure nitrogen gas to create a local anaerobic environment. The method, often referred to as the Hungate technique, named after its developer, is effective for liquid cultures, but less so with solid cultures, because this method employs a conduit to introduce a stream of N₂ gas for replacement of the head space gas inside of the tubes or flasks, while a bent needle or capillary is used for streaking or picking up colonies. To have a bent inoculating tool passing through the narrow opening of the flask without touching the conduit is extremely challenging, which raises the concern of frequent cross contamination. As a consequence, the conduit needs to be frequently sterilized, costing extra amount of time and resources.

Alternatively, Jaing et al. have developed an overlay technique where an inoculum is injected into a small volume of top agar in Hungate tubes. The cell-embedded top agar is then immediately transferred by syringe into flasks stored in an anaerobic chamber that already contained a bottom layer of media.

In view of these considerations, disclosed hereby is an improved method to prepare Thermotoga solid cultures and liquid cultures independent of an anaerobic chamber or conduit. In part, this disclosure describes an embedded cultivation method that greatly simplifies the cultivation methods previously used.

II. Subcloning, Characterization, and Use of a Thermotoga Restriction-Modification System:

Restriction endonucleases are enzymes that occur naturally in certain unicellular microbes—mainly bacteria and archaea—and that function to protect these organisms from infections by viruses and other parasitic DNA elements. Restriction endonucleases bind to specific sequences of nucleotides (‘recognition sequence’) in double-stranded DNA molecules (dsDNA) and cleave the DNA, usually within or close to the sequence, disrupting the DNA and triggering its destruction. Restriction endonucleases commonly occur with one or more companion enzymes termed modification methyltransferases.

Methyltransferases bond to the same sequences in dsDNA as the restriction endonucleases they accompany, but instead of cleaving the DNA, they alter it by addition of a methyl group to one of the bases within the sequence. This methylation ('modification') prevents the restriction endonuclease from binding to that site thereafter, rendering the site resistant to cleavage. Methyltransferases function as cellular antidotes to the restriction endonucleases they accompany, protecting the cell's own DNA from destruction by its restriction endonucleases. Together, a restriction endonuclease and its companion modification methyltransferase(s) form a restriction-modification (R-M) system, an enzymatic partnership that accomplishes for microbes what the immune system accomplishes, in some respects, for multicellular organisms. R-M systems are often strain-specific, allowing bacteria to differentially destroy invading DNA.

Thousands of R-M systems have been identified through massive screening of a large number of strains (Whitehead and Brown 1985; Hjorleifsdottir et al. 1996) or more recently by bioinformatic analysis of genome sequences (Matveyev et al. 2001; Ishikawa et al. 2005). A large and varied class of restriction endonucleases has been classified as ‘Type II’ restriction endonucleases. Type II systems are composed of just two proteins, R and M, and they usually act independently. They break DNA at or near the recognition sites at specific positions. The restriction activity requires Mg²⁺ but not ATP. These enzymes cleave DNA at defined positions, and in purified form, can be used to cut DNA molecules into precise fragments for gene cloning and analysis. The biochemical precision of Type II restriction endonucleases far exceeds anything achievable by chemical methods, making these enzymes the reagents sine qua non of molecular biology laboratories. In this capacity, as molecular tools for gene dissection, Type II restriction endonucleases have had a profound impact on the life sciences in the last 30 years, transforming the academic and commercial arenas alike. Their utility has spurred a continuous search for new restriction endonucleases, and a large number have been found. Today more than 200 Type II endonucleases are known, each possessing different DNA cleavage characteristics (Roberts and Macelis, Nucl. Acids Res., 29:268-69 (2001)). (REBASE®, http://rebase.neb.com/rebase). Concomitantly, the production and purification of these enzymes has been improved by the cloning and over-expression of the genes that encode them in non-natural production strain host cells such as E. coli.

Since the various restriction enzymes appear to perform similar biological roles, in much the same ways, it might be thought that they would resemble one another closely in amino acid sequence and behavior. Experience shows this not to be true, however. Surprisingly, far from resembling one another, most Type II restriction enzymes appear unique, resembling neither other restriction enzymes nor any other known kind of protein. Type II restriction endonucleases seem to have arisen independently of one another for the most part during evolution, and to have done so hundreds of times, so that today's enzymes represent a heterogeneous collection rather than a discrete family. Some restriction endonucleases act as homodimers, some as monomers, others as heterodimers. Some bind symmetric sequences, others asymmetric sequences; some bind continuous sequences; others, discontinuous sequences; some bind unique sequences, others multiple sequences. Some are accompanied by a single methyltransferase, others by two, and yet others by none at all. When two methyltransferases are present, sometimes they are separate proteins, at other times they are fused. The orders and orientations of restriction and modification genes vary, with all possible organizations occurring. Several kinds of methyltransferases exists, some methylating adenines (m6A-MTases), others methylating cytosines at the N-4 position (m4C-MTases), or at the 5 position (m5C-MTases). Usually there is no way of predicting, a priori, which modifications will block a particular restriction endonuclease, which kinds of methyltransferases will accompany that restriction endonuclease in any specific instance, nor what their gene orders will be.

From the point of view of cloning a Type II restriction endonuclease, the great variability that exists among restriction-modiciation systems means that, for experimental purposes, each is unique. Each enzyme is unique in amino acid sequence and catalytic behavior; each occurs in unique enzymatic association, adapted to unique microbial circumstances; and each presents the experimenter with a unique challenge. Sometimes a restriction endonuclease can be cloned and over-expressed in a straightforward manner, but more often than not it cannot, and what works well for one enzyme can work not at all for the next. Success with one is no guarantee of success with another. Because there exists an increasing demand for tractable tools to enable genetic analyses and manipulations of Thermotoga for the reasons described above, understanding the R-M systems of Thermotoga is a necessary step towards genetically modifying these organisms. A technical obstacle to any genetic engineering effort is the restriction-modification (R-M) systems of the host. If not properly modified, foreign DNA molecules will likely be restricted by host endonucleases as soon as they enter the new cell. Thus, the examples below also include the use of the cloned Thermotoga methyltransferase in genetic manipulation studies.

Based on sequence comparison to related genes, the Restriction Enzyme Database (REBASE) (Roberts et al. 2010) predicts there are three methyltransferase genes in the genome of T. neapolitana: CTN_{—}0340, CTN_{—}1203, and CTN_{—}1590. It further suggests that CTN_{—}0339 and CTN_{—}0340 constitute a Type II R-M system recognizing CGCG sequences with an unclear cleavage site. In the NCBI database, CTN_{—}0339 is annotated as a hypothetical gene, and CTN_{—}0340 as an m⁴C-MTase gene. These two genes are clustered on the chromosome with a convergent orientation. The examples discussed below act to validate the functional assignments of the two genes made by REBASE and to facilitate the construction of genetic tools for Thermotoga.

In part, this disclosure describes the subcloning, characterization, and use of a Thermotoga Restriction-modification system.

III. Genetic Manipulations of Thermotoga spp.:

Cryptic mini-plasmids pRQ7, pMC24, and pRKU1 have been identified in T. sp. RQ7, T. maritima, and T. petrophila RKU-1, respectively. Although the species from which they arise were discovered at geologically unrelated locations, the three plasmids are nearly identical. They differ by no more than three point mutations, all are extremely small (846 bp), and encode just one apparent open reading frame, presumably the replication protein. Studies of pRQ7 suggest that the plasmid is negatively supercoiled and replicates by a rolling-circle mechanism. Based on pRQ7, two Thermotoga-E. coli shuttle vectors pJY1 (chloramphenicol-resistant) and pJY2 (kanamycin-resistant) have been constructed for expression in T. neapolitana and T. maritima, respectively, as described by Yu et al. (2001)). Through liposome-mediated transformation, both vectors rendered transient antibiotic resistance to Thermotoga cells in liquid media, but no transformants could be isolated from plates. To date, that report remains the only documented effort of expressing heterologous genes in Thermotoga, out of more than 1200 publications retrieved from PubMed using “Thermotoga” as the key word (last searched Jun. 16, 2011). In fact, genetic manipulation of Thermotoga remains a challenge. To develop a tractable gene transfer system for Thermotoga spp., every aspect pertaining to the cloning and expression of foreign genes in Thermotoga, from plating efficiency to vector stability was systematically examined. The examples below show that heterologous genes can be introduced to Thermotoga through multiple means, be functionally expressed, and be stably maintained.

In part, this disclosure describes the creation and use of Thermotoga-E. coli shuttle vectors.

### SUMMARY

In one embodiment a new and improved culturing method for Thermotoga spp. is described.

In another embodiment, an isolated or recombinant DNA sequence coding for R.TneD1 or a functional derivative thereof is described. In another related embodiment, a vector comprising such DNA sequence is described, as is a host cell transformed with such a vector. In yet another related embodiment, a process for the manufacture of R.TneD1 or functional derivative thereof comprising cultivation of a cells transformed with such a vector; and the R.TneD1 or functional derivative thereof prepared by such process.

In another embodiment, an isolated or recombinant DNA sequence coding for M.TneD1 or a functional derivative thereof is described. In another related embodiment, a vector comprising such DNA sequence is described, as is a host cell transformed with such a vector. In yet another related embodiment, a process for the manufacture of M.TneD1 or functional derivative thereof comprising cultivation of a cells transformed with such a vector; and the M.TneD1 or functional derivative thereof prepared by such process.

In yet another embodiment, a vector capable of replication in both Thermatoga and non-Thermotoga species is described.

### BRIEF DESCRIPTION OF THE DRAWINGS

The above, as well as other advantages of the present disclosure, will become readily apparent to those skilled in the art from the following detailed description when considered in the light of the accompanying drawings in which:

FIG. 1 shows single colonies formed by T. sp. RQ7 cells. (a) Embedded growth. Cells were mixed with hot SVO medium containing 0.3% GELRITE and were poured to Petri dishes before solidification. (b) Surface growth. Cells were spread evenly on the surface of freshly-made SVO plates containing 0.3% GELRITE and 0.7% agar. The number on each plate indicates the dilution factor of each culture.

FIG. 2 shows XL1-Blue MRF′ recombinant strains grown at 30° C. (A), 37° C. (B), or 42° C. (C).

FIG. 3 shows digestion of pUC19 DNA with extracts of XL1-Blue MRF′ carrying pJC339 at 50° C. (A), 65° C. (B), or 77° C. (C). The amount of cell extract per μg DNA is labeled on top of each lane (in μl). M, λ/HindIII. Analyzed with 1% (w/v) agarose gel.

FIG. 4 shows R.TneDI-digested pUC19 DNA analyzed with 2% (w/v) agarose gel. Sizes of the fully digested fragments match the occurrence and locations of CGCG sites in the plasmid.

FIG. 5 shows protection of pUC19 DNA by M.TneDI. (A) Plasmid DNA was treated with various amount of cell extract of XL1-Blue MRF′ expressing CTN_{—}0340, as labeled on top of each lane. The DNA was then subjected to the digestion of R.TneDI (0.1 U per μg DNA). (B) Plasmid DNA was treated with various amount of cell extract of XL1-Blue MRF′ expressing both CTN_{—}0340 and CTN_{—}0339. The amount of cell extract per μg DNA is labeled on top of each lane (in μl).

FIG. 6 shows. SDS-PAGE (top) and western blotting (bottom) analyses of extracts of BL21(DE3) carrying both pJC339 and pJC340. Arrows point to the position of R.TneDI. Cells carrying the parent plasmids pET-24a(+) and pJC184 were used as the control.

FIG. 7 shows R.TneDI cleaved at the center of its recognition sequence (CG↓CG). (A) The sequence of the smaller fragment of CTN_{—}0339/R.TneDI ended at CG. The extra A at the 3′-end, donated by an asterisk, was a template-independent addition by Taq polymerase (Clark 1988; Stier and Kiss 2010). (B) The larger fragment of CTN_{—}0339/R.TneDI was ligated with pUC19/SmaI with their blunt ends. The half recognition sites of the enzymes are underlined. The shaded nucleotides represent CTN_{—}0339 DNA sequences.

FIG. 8 shows sequence alignment of M.TneDI and M.PvuII generated by CLUSTAL W (1.81) (Thompson et al. 1994) and shaded by Boxshade 3.3.1. Conserved residues are highlighted in black and similar residues in gray. Nine possible structural motifs (IV-III), which are well conserved amongst Group β MTase (Malone et al. 1995), are identified in M.TneDI and are underlined.

FIG. 9 shows a genetic map of pDH21.

FIG. 10 depicts protection of pUC19 by M.TneDI prepared from strain DH1021-3. M, Molecular maker λ/HindIII; 1, pUC19; 2-7, pUC19 treated with M.TneDI in the presence of 0, 20, 40, 80, 160, and 320 μg/ml AdoMet, respectively, and was digested with BstUI.

FIG. 11 is a genetic map of the shuttle vector pDH10. The region highlighted in bold represents the sequence of pRQ7.

FIG. 12 shows sensitivity of Thermotoga spp. to kanamycin. (a) Sensitive cells formed inhibition zones surrounding the paper discs loaded with various amounts of the antibiotic, as indicated in the top left panel. Gas bubbles produced by the Thermotoga cells were clearly visible in each plate. (b) Optical densities of T. maritima liquid cultures grown with kanamycin ranging from 0 to 300 μg/ml. Results of three independent tests. Tm, T. maritima; Tn, T. neapolitana; RQ7, T. sp. RQ7.

FIG. 13 shows detection of the transformed kan gene. PCR products of the kan gene were obtained from the plasmid extracts (P, labeled in bold) or the genomic DNA preparations (G) of the recombinant strains. Three RQ7/pDH10 and one Tm/pDH10 transformants, all obtained by electroporation, were examined. Plasmid extracts from DH5/pDH10 and T. sp. RQ7 were included as positive and negative controls, respectively. Analyzed with a 0.8% agarose gel.

FIG. 14 shows restriction digestions of the kan gene. PCR products of the kan gene were prepared from the plasmid extracts of DH5/pDH10 (lanes 1 and 4), RQ7/pDH10 (lanes 2 and 5), and Tm/pDH10 (lanes 3 and 6). M, 2-log DNA ladder. Analyzed with a 2% agarose gel.

FIG. 15 is a comparison of the copy numbers of pDH10 and pKT1 in E. coli. Plasmid DNA was extracted from the same amount of recombinant cells and was digested by XbaI and EcoRI. The arrow indicates the shared sequence of the two vectors. Analyzed with a 0.8% agarose gel.

### Sequence Listing

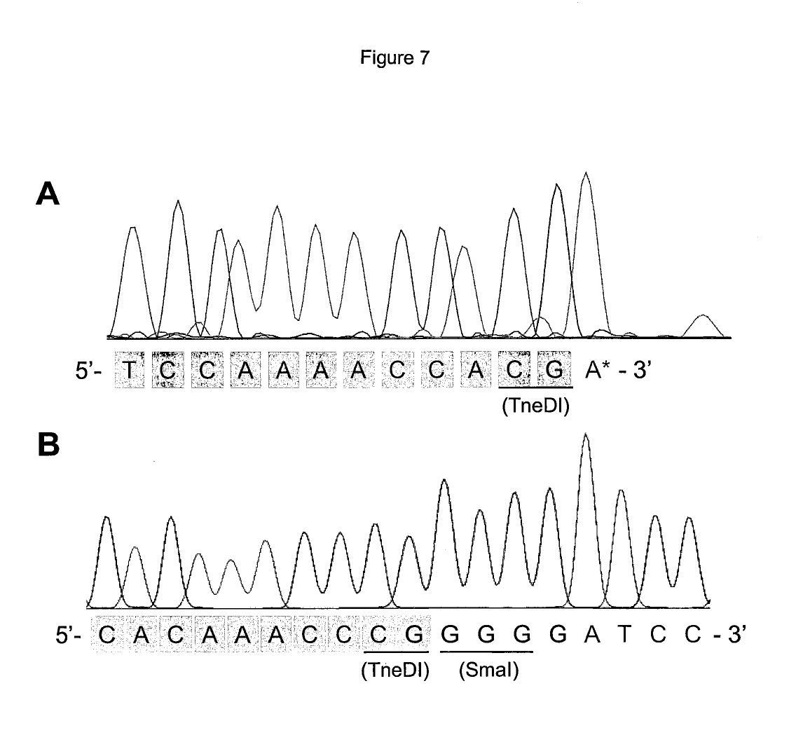

The nucleic and amino acid sequences listed in the accompanying sequence listing are shown using standard letter abbreviations for nucleotide bases, and three letter code of amino acids, as defined in 37 C.F.R. §1.822. Only one strand of each nucleic acid sequence is shown, but the complementary strand is understood as included by any reference to the displayed strand. In the accompanying sequence listing:
SEQ ID NO: 1 shows the nucleic acid sequence of pJC184.
SEQ ID NO: 2 shows the nucleic acid sequence of pDH22
SEQ ID NO: 3 shows the nucleic acid sequence of primerCTN_{—}0339 5′.
SEQ ID NO: 4 shows the nucleic acid sequence of primerCTN_{—}0339 3′.
SEQ ID NO: 5 shows the nucleic acid sequence of primer CTN0340 5′ inv.
SEQ ID NO: 6 shows the nucleic acid sequence of primer CTN0340 3′ inv.
SEQ ID NO: 7 shows the nucleic acid sequence of primer CTN_{—}0340 5′.
SEQ ID NO: 8 shows the nucleic acid sequence of primer CTN_{—}0340 3.′
SEQ ID NO: 9 shows the nucleic acid sequence of pJC339.
SEQ ID NO: 10 shows the nucleic acid sequence of pJC340.
SEQ ID NO: 11 shows the nucleic acid sequence of CTN_{—}0339.
SEQ ID NO: 12 shows the nucleic acid sequence of CTN_{—}0340.
SEQ ID NO: 13 shows the amino acid sequence of R.Tne.DI protein.
SEQ ID NO: 14 shows the amino acid sequence of M.Tne.DI protein.
SEQ ID NO: 15 shows the nucleic acid sequence of R.Tne.DI cut and plasmid ligation site as shown in FIG. 7A.
SEQ ID NO: 16 shows the nucleic acid sequence of R.Tne.DI cut and plasmid ligation site as shown in FIG. 7B.
SEQ ID NO: 17 shows the amino acid sequence of M.Pvu II protein.
SEQ ID NO: 18 shows the amino acid sequence of TSPPY/F conserved catalytic center.
SEQ ID NO: 19 shows the amino acid sequence of FxGxG/N conserved catalytic center.
SEQ ID NO: 20 shows the nucleic acid sequence of pDH10.
SEQ ID NO: 21 shows the nucleic acid sequence of pDH21.
SEQ ID NO: 22 shows the nucleic acid sequence of primer pKT1 5′.
SEQ ID NO: 23 shows the nucleic acid sequence of primer pKT1 3′.

### DETAILED DESCRIPTION

### A. Abbreviations

Ap: ampicillin; CFU: colony forming unit; DNA: deoxyribonucleic acid; EDTA: ethylenediaminetetraacetic acid; Kan: kanamycin; LB: Luria Broth; PCR: polymerase chain reaction; Tm: Thermotoga maritima; Tn: Thermotoga neapolitana; RQ7; Thermotoga sp. RQ7.

### B. Terms

Unless otherwise noted, technical terms are used according to conventional usage. Definitions of common terms in molecular biology may be found in Benjamin Lewin, Genes V., published by Oxford University Press, 1994 (ISBN 0-19-854287-9); Kendre et al. (eds.), The Encyclopedia of Molecular Biology, published by Blackwell Science Ltd., 1994 (ISBN 0-632-2182-9); and Robert A. Meyers (ed.), Molecular Biology and Biotechnology: a Comprehensive Desk Reference, published by VCH Publishers, Inc. 1995 (ISBN 1-56081-569-8). As also noted below, these publications are hereby incorporated to the extent permitted by law.

The following explanations of terms and methods are provided to better describe the present compounds, compositions, and methods and to guide those of ordinary skill in the art in the practice of the present disclosure. It is also to be understood that the terminology used in the disclosure is for the purpose of describing particular embodiments and examples only and is not intended to be limiting.

As used in the specification and the appended claims, the singular forms “a,” “an” and “the” include plural referents unless the context clearly dictates otherwise. Thus, for example, “a cell” may refer to a population of cells.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint.

As used herein the definition “methyltransferase or functional derivative thereof” refers to enzymes which have the capability of methylating a specific DNA sequence, as described below. Accordingly, the definition embraces all non-naturally occurring methyltransferases having such capability including functional variants, such as functional fragments, mutants resulting from mutagenesis or other recombinant techniques. It is not intended to include other naturally occurring methyltransferases that methylate the same DNA sequence. Furthermore, the definition is intended to include glycosylated or unglycosylated methyltransferases, polymorphic or allelic variants and other isoforms of the enzyme. “Functional derivatives” of the enzyme can include functional fragments, functional fusion proteins or functional mutant proteins.

Such methyltransferases included in the present invention can have a deletion of one or more amino acids, such deletion being an N-terminal, C-terminal or internal deletion. Also truncated forms are envisioned as long as they have the conservation capability indicated herein.

As used herein the definition “restriction endonuclease or functional derivative thereof” refers to enzymes which have the capability of cutting the specific DNA sequence, as described below. Accordingly, the definition embraces non-naturally occurring restriction endonucleases having such capability including functional variants, such as functional fragments, mutants resulting from mutagenesis or other recombinant techniques. It is not intended to include other naturally occurring restriction endonucleases that cut the same DNA sequence. Furthermore, the definition is intended to include glycosylated or unglycosylated restriction endonucleases polymorphic or allelic variants and other isoforms of the enzyme. “Functional derivatives” of the enzyme can include functional fragments, functional fusion proteins or functional mutant proteins. Such restriction endonucleases included in the present invention can have a deletion of one or more amino acids, such deletion being an N-terminal, C-terminal or internal deletion. Also truncated forms are envisioned as long as they have the conservation capability indicated herein.

Operable fragments, mutants or truncated forms can suitably be identified by screening. This is made possible by deletion of for example N-terminal, C-terminal or internal regions of the protein in a step-wise fashion, and the resulting derivative can be analyzed with regard to its capability of the desired methyltransferase or restriction endonuclease activity. If the derivative in question operates in this capacity it is considered to constitute a functional derivative of the methyltransferase or restriction endonuclease proper.

C. Reference will now be made in detail to the present preferred embodiments. Some examples of which are illustrated in the accompanying drawings. Whenever possible, the same reference numbers are used throughout the drawings to refer to the same or like parts.

I. Method for Preparation and Handling Solid Cultures of Thermotoga Spp. Under Aerobic Conditions:

### Improved Method for Cultivation of Thermotoga.

The success of isolating transformants from solid media is essential to any genetic manipulation attempt. Whereas this is not a concern with aerobic mesophiles like E. coli, this requirement has become a limiting factor for the genetic investigations of many strict anaerobic, hyperthermophilic organisms. One obstacle is the requirement of an anaerobic glove box to handle plates. Since picking up colonies requires great precision, reaching out to a single colony with an inoculation loop or a toothpick through thick gloves has proven to be challenging for many of us. Even though gloveless chambers are commercially available, they are costly to maintain. Rolling tubes or tissue culture flasks in combination to Hungate techniques may serve as alternatives, but they are prone to cross contaminations due to the narrow openings of these containers. Based on the fact that Thermotoga are fairly oxygen-tolerant, especially when they are not actively growing, we prepare Thermotoga solid cultures with an embedded method, independent of an anaerobic chamber or an anoxic gas conduit. Our method sustains ˜50% plating efficiency, making it is possible to select for Thermotoga transformants among a sizeable population of viable cells. In addition, we developed a soft SVO medium to bridge the transfer of cultures from solid media to liquid media in an aerobic environment. Soft SVO is easy to make and convenient to use, and it also allows the withdrawal of cultures using a syringe.

Thermotoga were cultivated at 77° C. in SVO medium developed by van Ooteghem et al. (2002). Fifty milliliters of SVO was dispensed into 100 ml serum bottles and sparged with nitrogen gas to remove oxygen from the medium and the headspace. Serum bottles were then sealed by rubber stoppers, secured by aluminum caps, and sterilized. Inoculation of the liquid SVO was done by a syringe needle with a typical inoculum of 2%. Liquid cultures were shaken at 100 rpm. For preparation of soft SVO, 0.075% agar was dissolved in liquid SVO. Culture tubes with screw caps were filled with soft SVO up to two thirds of the volume capacity and were autoclaved. To grow Thermotoga on plates, double strength (2×) of liquid SVO and various concentrations of agar or GELRITE (Sigma-Aldrich Co., St. Louis, Mo., USA) were autoclaved separately and then mixed with equal volumes while they were still hot. The medium either was directly poured to Petri dishes for standard spreading or streaking, or was mixed with cell cultures prior to pouring for embedded growth. A VACU-QUIK jar (Almore International Inc., Portland, Oreg., USA) containing a packet of 4 g of palladium catalyst was used for anaerobic cultivation of plates. The atmosphere inside of the jar was exchanged to 96:4 N₂—H₂ before it was placed to an incubator of 77° C. Colonies usually appear in 24 h and grow bigger in 48 h. Kanamycin was supplemented when needed at 150 μg ml⁻¹ for liquid and 250 μg ml⁻¹ for soft and solid cultures. Cell growth in liquid was monitored by measuring the optical density of cell cultures at 600 nm (OD₆₀₀). All aforementioned operations were carried out on the bench top.

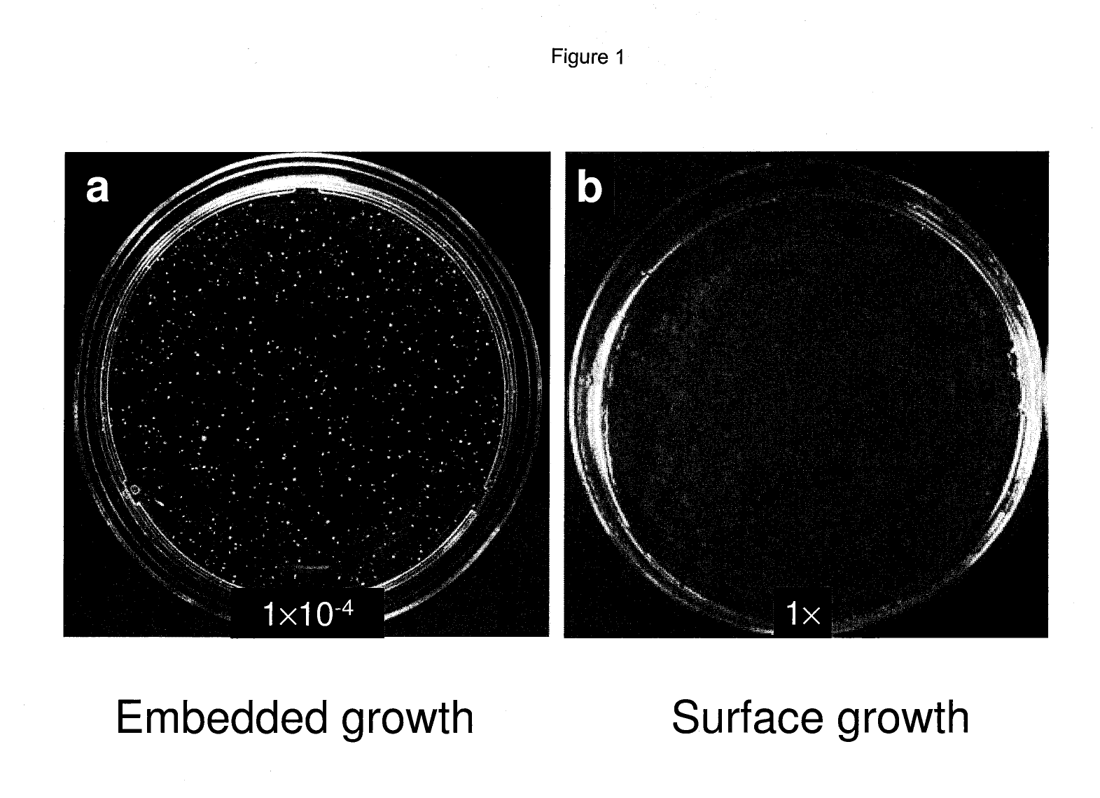

The chance of obtaining Thermotoga transformants on plates can be seriously compromised if plating efficiencies are low. Considering that Thermotoga can tolerate brief exposures to oxygen, we simplified the overlay methods used by other groups (Jiang et al. (2006)) and developed an embedded growth method. Properly diluted liquid cultures were suspended in hot SVO containing 0.3% GELRITE, and the mixtures were allowed to solidify in Petri dishes. In this method, cells were embedded in the medium matrix, and their exposure to oxygen was reduced. Ten microliters of an overnight culture of T. sp. RQ7 with a dilution factor of 10⁻⁴ formed 1256 colonies (FIG. 1a), which is equal to 1.26×10⁹ colony forming units (CFU) per ml. By contrast, a surface culture, prepared by standard spreading in the same environment, would typically generate 7.56×10³ CFU ml⁻¹ (FIG. 1b), about ten thousand times less. Given that T. neapolitana cultures contain approximate 3.0×10⁹ cells after growing in liquid SVO for 14 h at 77° C. (van Ootegham et al. (2004)) we estimate that the plating efficiency of our embedded method is close to 50%. This high efficiency enables us to select or screen a large number of single colonies while still enjoying the convenience of aerobic handling.

To facilitate the transfer of single colonies from solid to liquid media under aerobic conditions, we introduced a soft SVO medium by adding 0.075% agar to liquid SVO. Agar, or another solidifying agent, prevents atmospheric oxygen from penetrating deep into the medium and the reducing agent in the medium reduces dissolved oxygen to water. To transfer cultures from solid to soft SVO, single colonies were picked up from plates by a loop and were pushed down to the bottoms of the test tubes containing soft SVO, where a local anaerobic environment has been created. After 12-24 h of incubation, cultures grown in soft SVO were then transferred to liquid SVO by a syringe. Although the introduction of soft SVO seemed to prolong the overall operation cycle, it ensured maximum viability of Thermotoga cells during the transfer, which eventually allowed us to isolate Thermotoga transformants for the first time (see below). Cultures kept at the bench top for 2 months were still vital and exhibited no growth defects.

Although reference is made to agar or GELRITE, any solidifying agent known or obvious to one skilled in the art may be used in accordance with the invention. Also, although reference is made to SVO media in the detailed embodiments, any media known or obvious to one skilled in the art that supports the growth of Thermotoga spp. May be used in accordance with the invention. Preferably, The media will contain a reducing agent. To clarify what is meant by solid media, liquid media, and soft media: Solid media will have a viscosity greater than both that of soft media and that of liquid media. Liquid media will have a viscosity less than both that of solid media and soft media. Soft media will have a viscosity less than that of solid media, but greater than that of liquid media. While reference is made to specific vessels, e.g. test tubes, petri dishes, VACU-QUICK jars, and the like, any vessel known to be suitable for supporting the growth of bacteria under the conditions described is intended to be within the scope of the invention. Likewise, any known method for creating anaeraobic conditions when needed as indicated above, may be used without departing from the scope of the invention.

II. Subcloning, Characterization, and Use of a Thermotoga Restriction-Modification System:

### Strains and Cultivation Conditions

Bacterial strains and vectors involved in this study are listed in Table 1. T. neapolitana ATCC 49049 (same as DSM 4359) was obtained from the American Type Culture Collection (http://www.atcc.org/) and was cultivated at 77° C. in liquid SVO medium (Van Ooteghem et al. 2002). All DNA manipulations were carried out in E. coli XL1-Blue MRF′, and the expression of CTN_{—}0339 and CTN_{—}0340 was studied in both XL1-Blue MRF′ and E. coli BL21(DE3). E. coli strains were cultivated at 30, 37, or 42° C. in Luria-Bertani (LB) medium (1% tryptone, 0.5% NaCl, 0.5% yeast extract) with 1.5% (w/v) agar for plates. Kanamycin and chloramphenicol were added when needed at 50 μg ml⁻¹ and 30 μg ml⁻¹, respectively. Growth of E. coli strains was measured as optical density at 600 nm (OD₆₀₀). For BL21(DE3) recombinant strains, 0.1 mM IPTG (isopropyl-D-thiogalactopyranoside) was added to induce the expression of CTN_{—}0339 when culture density reached about 0.4 to 0.6. Induced cultures were incubated for another 4 h prior to further analyses.

Cloning of CTN 0339 into pET-24a(+)

The complete genome sequence of T. neapolitana is available at http://www.ncbi.nlm.nih.gov/genomeprj/21023. A DNA fragment corresponding to the coding region of CTN_{—}0339 (672 bp, excluding the stop codon) was amplified from the genomic DNA of T. neapolitana with primers 5′-GGAATTCCATATGAGAAAAACGGATCCTCTCAT-3′, which allows the start codon to be embedded in an NdeI recognition site, and 5′-CACAAGCTTCTGTTGATATTTTTCTATCA-3′, which introduces a HindIII site at the end of sequence. The PCR product (691 bp, including NdeI and HindIII linkers) was purified by ethanol precipitation, digested with the two restriction enzymes, and inserted to the same sites of the E. coli expression vector pET-24a(+) to generate pJC339. The expressed protein has an appended His tag for immunodetection.

It is within the scope of the invention to amplify CTN_{—}0339 coding region with primers that will allow for subsequent insertion into any suitable vector for expression of the CTN_{—}0339 gene products in E. coli or other host bacteria. Further, any known epitope tag for later immunodetection of the CTN_{—}0339 gene product is contemplated to be within the scope of the invention. The invention also contemplates any DNA sequence with at least about 95% sequence identity with CTN_{—}0339, more preferably at least about 90% sequence identity with CTN_{—}0339, even more preferably at least about 85% sequence identity with CTN_{—}0339, still even more preferably at least about 80% sequence identity with CTN_{—}0339, and most preferably at least about 75% sequence identity with CTN_{—}0339.

The invention also contemplates any DNA sequence with at least about 95% sequence identity with pJC339, more preferably at least about 90% sequence identity with pJC339, even more preferably at least about 85% sequence identity with pJC339, still even more preferably at least about 80% sequence identity with pJC339, and most preferably at least about 75% sequence identity with pJC339.

It is not beyond the scope of the invention to insert subset or subsets of the CTN_{—}0339 DNA sequence that may code for functional regions of the resulting protein, rather than the entire CTN_{—}0339 DNA sequence.

Cloning of CTN 0340 into pACYC184

CTN_{—}0340 was cloned to pACYC184 under the control of the promoter for the tetracycline resistance gene (tet). First the coding region of the tet gene as well as its downstream region (1581-3603) was removed from plasmid pACYC184 by inverse PCR using primers 5′-GAATTCCATATGCGGTGCCTGACTGCGTTAGC-3′ and 5′-GAATTCCATATGGAATTCCCGCGGATCCTGAGAAGCACACGGTCACACTGCTTCC-3′. The PCR product carries an NdeI site at its 5′ end and a BamHI-NdeI linker at its 3′ end. After digestion with NdeI, the fragment was allowed to recircularize to form pJC184. Meanwhile, primers 5′-GAATTCCATATGAAGAGAAGAAAATCAACAAG-3′ and 5′-CGCGGATCCTTATCAACAGTGATCTTCAA-3′ were used to amplify the 927 bp coding region of CTN_{—}0340 from the chromosome of T. neapolitana. The primers were designed to have the start codon embedded in an NdeI site and the stop codon followed by a BamHI site. PCR fragment of CTN_{—}0340 was then digested with NdeI and BamHI and was inserted to the corresponding sites of pJC184 to generate pJC340.

It is within the scope of the invention to amplify CTN_{—}0340 coding region with primers that will allow for subsequent insertion into any suitable vector for expression of the CTN_{—}0340 gene products in E. coli or other host bacteria. Further, any known epitope tag for later immunodetection of the CTN_{—}0340 gene product is contemplated to be within the scope of the invention. The invention also contemplates any DNA sequence with at least about 95% sequence identity with CTN_{—}0340, more preferably at least about 90% sequence identity with CTN_{—}0340, even more preferably at least about 85% sequence identity with CTN_{—}0340, still even more preferably at least about 80% sequence identity with CTN_{—}0340, and most preferably at least about 75% sequence identity with CTN_{—}0340.

The invention also contemplates any DNA sequence with at least about 95% sequence identity with pJC340, more preferably at least about 90% sequence identity with pJC340, even more preferably at least about 85% sequence identity with pJC340, still even more preferably at least about 80% sequence identity with pJC340, and most preferably at least about 75% sequence identity with pJC340.

It is not beyond the scope of the invention to insert subset or subsets of the CTN_{—}0340 DNA sequence that may code for functional regions of the resulting protein, rather than the entire CTN_{—}0340 DNA sequence.

### Purification and Analyses of CTN 0339 and CTN 0340 Gene Products

One gram of E. coli wet cells grown at 37° C., containing pJC339 or pJC340 or both, were resuspended in 3 ml lysis buffer (10 mM Tris-HCl, 10 mM MgCl₂, 50 mM NaCl, 1 mM DTT, pH 8.0) and were lysed with two passages through a French press at 14,000 pounds per square inch. After centrifugation at 27 k×g for 25 min, the supernatant was transferred to a new tube and heated at 77° C. for 1 h to denature most of the host proteins, followed by another round of centrifugation at 17 k×g for 15 min. The supernatant containing purified Thermotoga proteins was stored at 4° C. Such extracts retained 100% of the restriction activity for up to 5 weeks, and ≧50% of the activity for at least 12 weeks. For expression analysis, heat-purified cell extracts were mixed with loading buffer and were subjected to sodium dodecyl sulfate-polyacrylamide gel electrophoresis (SDS-PAGE) in 15% (w/v) gels. HisDetector Western Blot Kit (Kirkegaard & Perry Laboratories, Inc., Maryland, USA) was used for colorimetric immunodetection of histidine-tagged CTN_{—}0339 protein. For restriction assays, heat-purified cell extracts containing the REase were serially diluted in 2-fold steps with the lysis buffer and were incubated with 5 μg of substrate DNA in a reaction volume of 50 μl. After 1 h of incubation at 50° C., 65° C., or 77° C., the reaction was terminated with 5 μl of stopping buffer (50 mM EDTA, 50% (w/v) glycerol, 0.02% (w/v) bromophenol blue, pH 8.0). Digested DNA fragments were resolved on 1% (w/v) agarose gels. One unit of the REase activity is defined as the quantity required to completely digest 1 μg of pUC19 DNA in 1 h at 77° C. For modification assays, heat-purified cell extracts containing the MTase were serially diluted in 2-fold steps with the lysis buffer and were incubated with 5 μg of substrate DNA in a total volume of 50 μl. After 1 h of incubation at 77° C., the reaction mix was then incubated with 0.5 U of the REase at 77° C. for overnight. One unit of the MTase activity is defined as the quantity required to completely protect 1 μg of pUC19 DNA from the cleavage of the REase for 1 h at 77° C.

The invention contemplates R.TneD1 and/or M.TneD1 may contain an epitope tag.

The invention also contemplates any amino acid sequence with at least about 95% sequence identity with R.TneD1 or its functional derivatives, more preferably at least about 90% sequence identity with R.TneD1 or its functional derivatives, even more preferably at least about 85% sequence identity with R.TneD1 or its functional derivatives, still even more preferably at least about 80% sequence identity with R.TneD1 or its functional derivatives, and most preferably at least about 75% sequence identity with R.TneD1 or its functional derivatives. The sequence identity will be measured without consideration of any epitope tag that may be joined to the R.TneD1 protein proper.

The invention also contemplates any amino acid sequence with at least about 95% sequence identity with M.TneD1 or its functional derivatives, more preferably at least about 90% sequence identity with M.TneD1 or its functional derivatives, even more preferably at least about 85% sequence identity with M.TneD1 or its functional derivatives, still even more preferably at least about 80% sequence identity with M.TneD1 or its functional derivatives, and most preferably at least about 75% sequence identity with M.TneD1 or its functional derivatives. The sequence identity will be measured without consideration of any epitope tag that may be joined to the M.TneD1 protein proper.

### Determination of the Cleavage Site of the REase

The PCR product of CTN_{—}0339 was completely digested by heat-purified REase. Two fragments were generated and were retrieved from agarose gels. The smaller piece was directly analyzed by Sanger sequencing; the larger piece was further digested by NdeI and was inserted into pUC19 at the NdeI-SmaI sites to form pDH22. The insertion region of the constructed vector was sequenced.

Growth of E. coli Recombinant Strains

Plasmids pJC339 and pJC340 were separately constructed in E. coli strain XL1-Blue MRF′ by screening the transformants at 30° C. The cloned sequence of CTN_{—}0339 (putative REase) and CTN_{—}0340 (putative MTase) were confirmed by both restriction digestions and DNA sequencing. Plasmid pJC339 was introduced to XL1-Blue MRF′/pJC340 to obtain strain XL1-Blue MRF′/pJC339+pJC340. On these plasmids, CTN_{—}0339 and CTN_{—}0340 are under the control of T7 and tet promoters, respectively. Unlike BL21(DE3), XL1-Blue MRF′ has no chromosomal T7 RNA polymerase gene, thus CTN_{—}0339 is transcribed merely at a basal level in an XL1-Blue MRF′ background. The tet promoter is constitutively active in most E. coli laboratory strains, including XL1-Blue MRF′ and BL21(DE3).

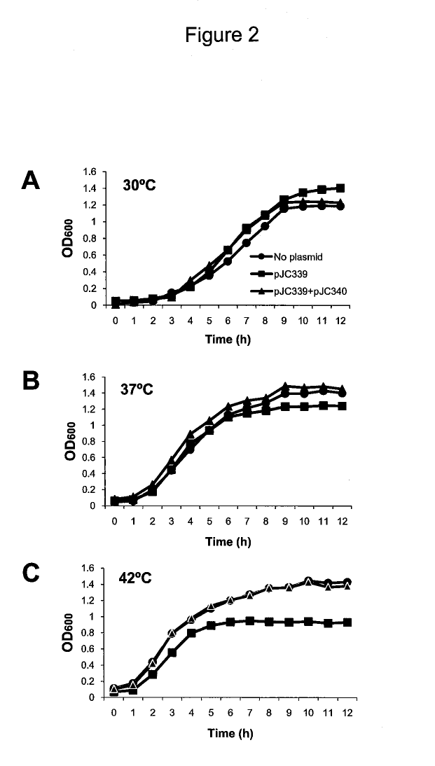

XL1-Blue MRF′ harboring pJC339 or along with pJC340 was cultivated at 30° C., 37° C., or 42° C., and the growth was monitored hourly. All strains grew equally well at 30° C. and 37° C. (FIG. 2) and displayed no signs of stress from expressing the Thermotoga proteins, which are not expected to be much active at these temperatures. At 42° C., cells carrying pJC339 and pJC340 propagated as well as the control strain did. With pJC339, however, the growth ceased at a low culture density. Therefore, at elevated temperatures, CTN_{—}0339 inhibits the physiological well-being of host cells, but this inhibition is countered by CTN_{—}0340. These observations are in agreement with the speculated restriction and modification activities of the two proteins.

This invention also contemplates bacterial strains containing any of the described herein DNA sequences coding for R.TneD1 or functional derivatives thereof and/or coding for coding for M.TneD1 or functional derivatives thereof.

### Restriction and Modification Assays of the Thermotoga Proteins

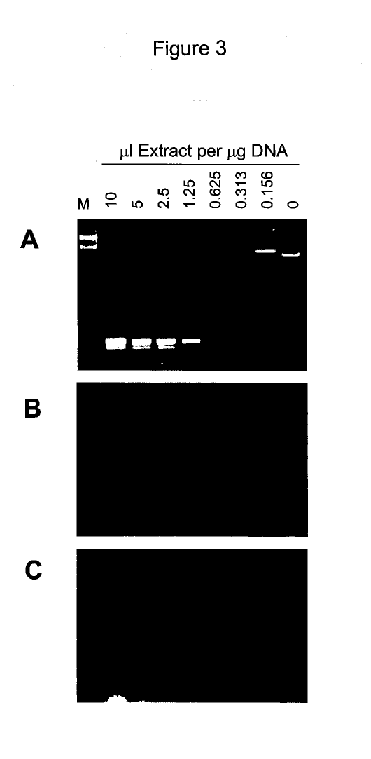

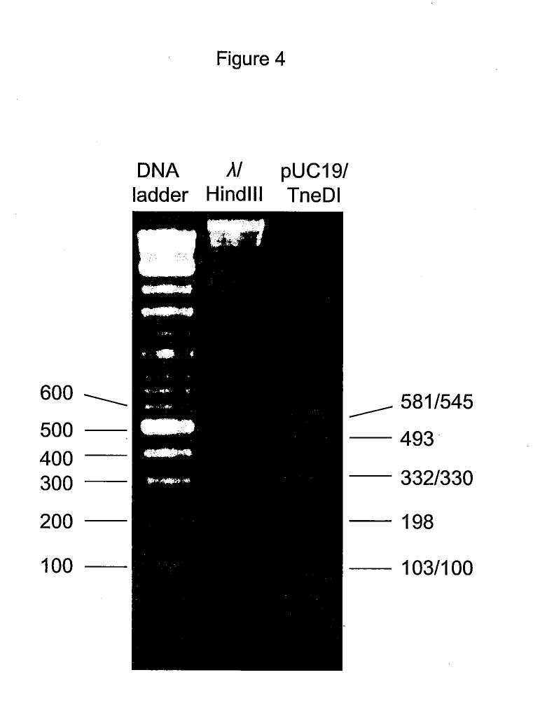

To provide further evidence to the functions of the two Thermotoga proteins, we first tested the restriction activity of CTN_{—}0339 at 50° C., 65° C., and 77° C. using pUC19 plasmid DNA prepared from strain XL1-Blue MRF′. Apparent REase activity was observed in cells expressing pJC339 at all tested temperatures, and the highest cleavage efficiency occurred at 77° C. (FIG. 3). For example, 2.5 μl of CTN_{—}0339 extract was required to completely digest 1 μg of pUC19 DNA within 1 h at 65° C. or 50° C., whereas only half of the amount was needed at 77° C. (FIG. 3). The sizes of fully digested pUC19 fragments agree with the occurrence and locations of CGCG sites in this plasmid (FIG. 4).

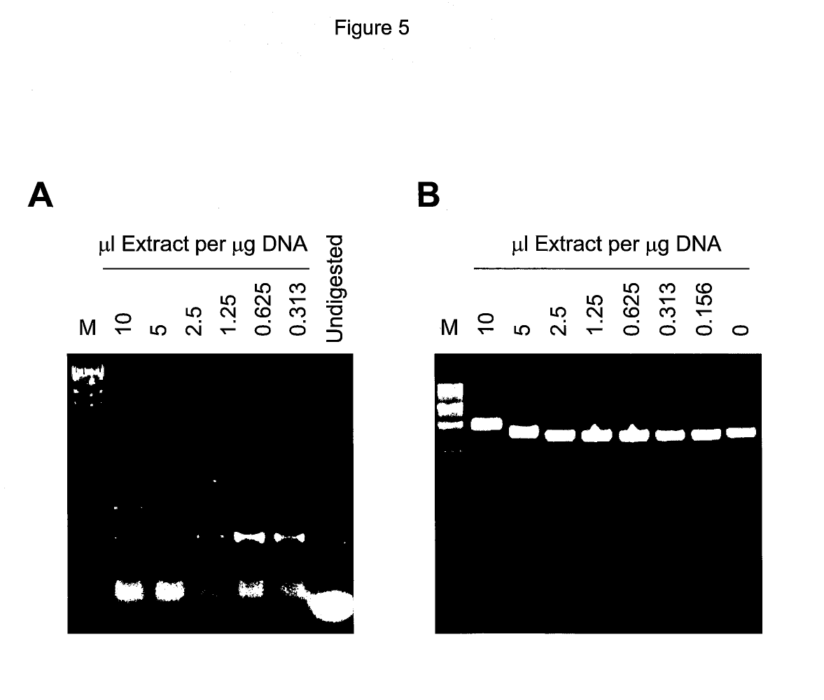

We next tested whether CTN_{—}0340 was able to protect pUC19 DNA from the digestion of the REase. The substrate DNA was incubated with CTN_{—}0340 extract at 77° C. for 1 h and then digested by the REase. DNA treated with 10 μl of CTN_{—}0340 extract was only partially digested by the REase, even after overnight incubation at 77° C. (FIG. 5A). If the CTN_{—}0340 extract was diluted in 2-fold steps, the substrate DNA became more and more susceptible to the same amount of R.TneDI, and full digestion eventually occurred (FIG. 5A). In another experiment, cell extract containing both CTN_{—}0339 and CTN_{—}0340 was used to treat pUC19 DNA (FIG. 5B). After 1 h of incubation at 55° C., the majority of the DNA was still intact (FIG. 5B), whereas under the same conditions, cell extract containing just CTN_{—}0339 would have substantially degraded the DNA (FIG. 3A). Low levels of partial digestion were noticed in some samples in FIG. 5B, reflecting a competition between the antagonistic activities of CTN_{—}0339 and CTN_{—}0340. Taken together of the above findings (FIGS. 2-5), it is conclusive that CTN_{—}0339 encodes the REase R.TneDI and CTN_{—}0340 the MTase M.TneDI.

### Overexpression of R.TneDI

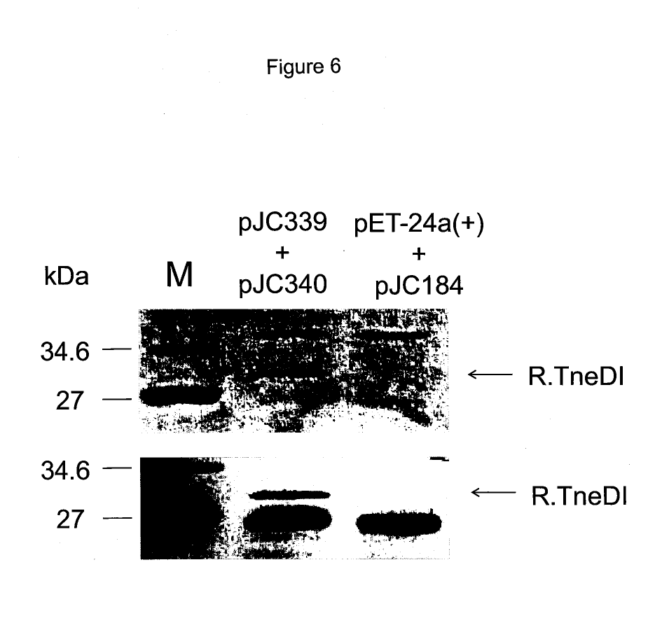

Although the REase activity was detected in XL1-Blue MRF′/pJC339, we were unable to observe the R.TneDI protein with either SDS-PAGE or immunoblotting, probably due to its extremely low expression level. To prepare a large quantity of R.TneDI for further applications, pJC340 and pJC339 were co-transformed to BL21(DE3), and cell extract of the recombinant strain was prepared and analyzed by SDS-PAGE. A distinctive band with an apparent size of ˜29 kDa was revealed (FIG. 6, top). Western blotting analysis confirmed that the specific band was the histidine-tagged R.TneDI (FIG. 6, bottom), which has a theoretical molecular weight of 28.3 kDa. Thus, it is feasible to overproduce R.TneDI in an E. coli host and subsequently purify it with an affinity column. The synthesis of M.TneDI, which has a theoretical molecular weight of 35.3 kDa, was below the detection limit of SDS-PAGE in both hosts. This is not surprising though, since pACYC184 is a low copy number vector and the tet promoter is not typically used for overexpression. Immunoblotting for this MTase was not attempted.

### Determination of the Cleavage Site of R.TneDI

Sequence analysis uncovered a recognition site (CGCG) in the coding region of R.TneDI. The PCR product of this region was digested by R.TneDI, generating an upstream fragment of 502 bp and a downstream fragment of 189 bp. Sequencing of the smaller piece revealed that R.TneDI cut immediately after the first G in its recognition sequence (CG↓CG) (FIG. 7A). To further confirm that R.TneDI is a blunt-end cutter, the larger fragment was digested with NdeI and was inserted into the NdeI-SmaI sites of pUC19. Sequencing of the new vector in the insertion region showed that the two DNA pieces, cut by R.TneDI and SmaI respectively, were joined together seamlessly (FIG. 7B).

In this example, we demonstrated that genes CTN_{—}0339 and CTN_{—}0340 of T. neapolitana encode the TneDI Type II R-M system. R.TneDI may serve as a handy tool for specific cleavage of DNA molecules, and an E. coli strain containing M.TneDI should be a good host for preparing the DNA to be introduced to a Thermotoga host. Type II REases recognizing CGCG sequence have been discovered before, such as AccII, FnuDII, ThaI, BsuE, Bsh1236I, BepI, and MvnI (Lui et al. 1979; Strobl and Thompson 1984; Gaido and Strobl 1987; Gaido et al. 1988; Thomm et al. 1988; Venetianer and Orosz 1988), all of which cleave at the center of the recognition site and create blunt ends for their DNA substrates. A notable exception is Sell, which cleaves at the ends of the recognition site (LCGCG) (Miyake et al. 1992).

To protect host chromosomal DNA, REases usually have to be cloned in the presence of the cognate MTases (Howard et al. 1986). Alternatively, in vitro transcription and translation approaches could be used (Ishikawa et al. 2005). Here we were able to directly clone R.TneD1 to XL1-Blue MRF′ by imposing its expression under the control of T7 promoter. The fact that R.TneDI is less active at mesophilic temperatures probably also contributed to the survival of the host cells. Similar success has been achieved before with other thermophilic R-M systems, such as TaqI (Slatko et al. 1987).

Thus, it is not beyond the boundaries of this invention to include host strains transformed with genetic material encoding for the R.TneD1 or M.TneD1 proteins or their functional derivatives.

In a modification reaction, AdoMet (S-adenosyl-L-methionine) serves as the methyl donor and is thus an essential cofactor for methylases. In our modification assays (FIG. 5), supplementation of AdoMet was not attempted, because the E. coli cell extracts were expected to carry enough of the cofactor, even after the heat treatment. Contrary to our common belief, AdoMet is reasonably stable in solutions. For instance, if crude yeast extract is incubated at 37° C., ˜70% of the original AdoMet can be found after 6 days and ˜25% for 15 days (Morana et al. 2002). Therefore, a fair amount of AdoMet is expected to survive after incubation at 77° C. for 1 h in the cell extract of E. coli. Moreover, our cell extracts were prepared from 1 g of wet cells (equivalent to 1 L at OD₆₀₀˜1.0) in 3 ml of lysis buffer. The cell density was extremely high. In our assays, the cell extracts were diluted for no more than 64 times. Consequently, even the most diluted sample should have enough AdoMet.

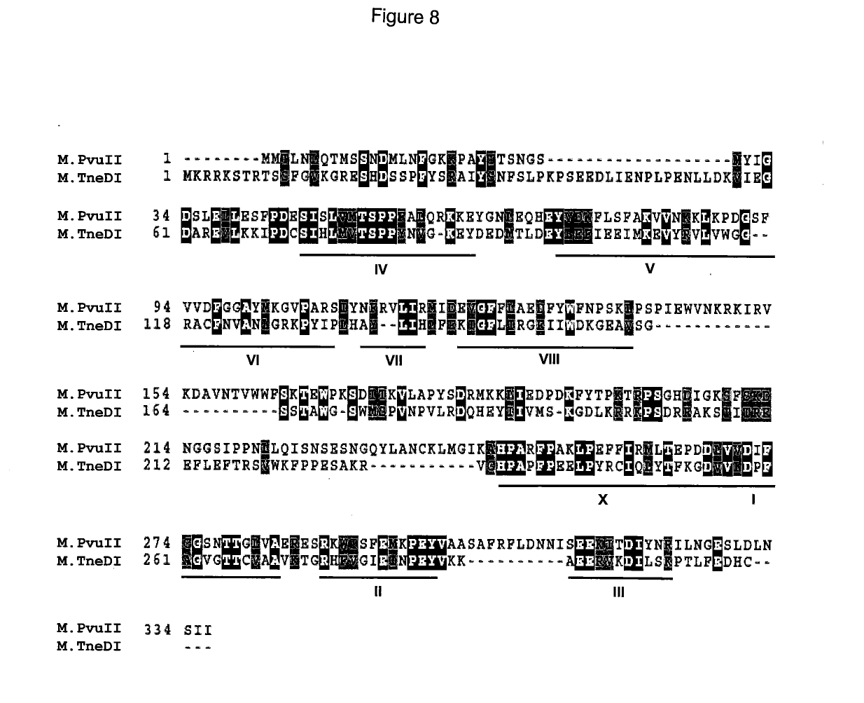

We have not determined which cytosine at the recognition site is modified by M.TneDI. Also unknown is the exact modification position of the cytosine, though motif analysis almost certainly identifies M.TneDI as an m⁴C MTase (Malone et al. 1995). Comparison of M.TneDI and M.PvuII, a m⁴C MTase with a known structure (Gong et al. 1997), revealed the conserved catalytic center (TSPPY/F) (in motif IV) and the Rossman fold serving as the AdoMet binding site (FxGxG/N) (in motif I) (FIG. 8). The alignment is more gapped in the target recognition domains, which is not surprising as PvuII system recognizes CAGCTG instead of CGCG. It has been suggested that m⁴C is more common in thermophiles than m⁵C, because an m⁵C residue has a higher risk of deamination at elevated temperatures (Ehrlich et al. 1985).

Orthologs of the TneDI system have been found in other members of the Thermotogaceae family. The locus IDs and identities of the orthologs pertaining to the TneDI system at the protein level are summarized in Table 2. The neighborhood genes in the six Thermotogaceae genomes share nearly perfect synteny. Of even greater interest, given the low level of sequence conservation among REases in general, is the presence of an orthologous R-M system in the archaeon Ferroglobus placidus. While F. placidus is phylogenetically distant from Thermotogaceae, they live in the same high-temperature anaerobic environment, suggesting a recent horizontal gene transfer event between the two groups.

### Construction and Screening of DH1021 Strains

A Thermotoga-specific restriction-modification system, TneDI, has been identified in most Thermotoga strains. To increase the transformation efficiency of foreign DNA, such as pDH10 (described below), into a Thermotoga strain, it is desirable to modify the foreign DNA with the methylase M.TneDI prior to the transformation attempt. Therefore, we decided to overexpress CTN_{—}0340 in E. coli BL21(DE3) for preparation of M.TneDI for in vitro methylation reactions. Construction of vector pDH21 and the DH1021 strains.

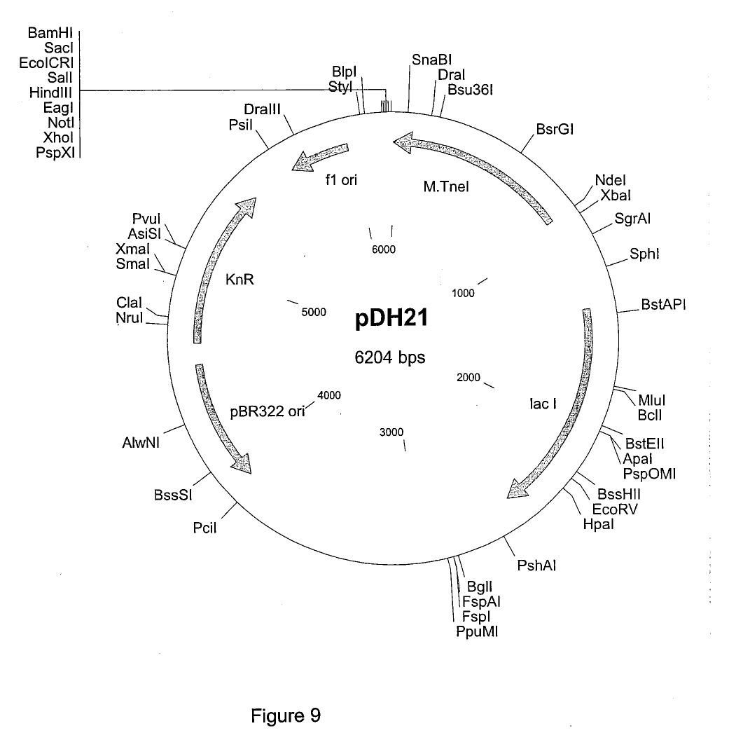

As described above, DNA fragment corresponding to the coding region of CTN_{—}0340 was obtained by digesting plasmid pJC340 with NdeI and BamHI. The NdeI-BamHI fragment was then joined to the expression vector pET-24a(+), which was pre-digested with the same enzymes, to generate pDH21 (FIG. 9). The ligation product was then transferred into E. coli strain XL1-Blue MRF′; the transformants were selected under 37° C. After verification, plasmid pDH21 was isolated from XL1-Blue MRF′/pDH21 and transferred into E. coli strain BL21(DE3); the transformants were selected at 30° C. Thirty one BL21(DE3)/pDH21 transformants (named as strains DH1021-1 to DH1021-31 hereafter) were obtained and were screened for their M.TneDI activity.

The invention also contemplates any DNA sequence with at least about 95% sequence identity with pDH21, more preferably at least about 90% sequence identity with pDH21, even more preferably at least about 85% sequence identity with pDH21, still even more preferably at least about 80% sequence identity with pDH21, and most preferably at least about 75% sequence identity with pDH21.

This invention also contemplates bacterial strains containing any of the described herein DNA sequences coding for R.TneD1 or functional derivatives thereof and/or coding for coding for M.TneD1 or functional derivatives thereof.

### Expression of M. TneDI.

Overnight culture of the DH1021 strains were transferred to fresh LB medium (1% tryptone, 0.5% NaCl, 0.5% yeast extract, 1.5% (w/v) agar) supplemented with 50 μg/ml kanamycin and were cultivated at 37° C. When the cell densities reached about 0.5 OD₆₀₀, IPTG was added to a final concentration 0.4 mM to induce the expression of M.TneDI. The cells were allowed to grow at 16° C. for overnight after the induction.

### Preparation and Assay of M. TneDI.

Cells from 10 ml of the induced DH1021 cultures were harvested by centrifugation and were resuspended in 150 μl of lysis buffer (10 mM Tris-HCl, 10 mM MgCl₂, 50 mM NaCl, 1 mM DTT, pH 8.0). Lysozyme was added to a final concentration of 0.3 mg/ml. The cell suspension was subject to 3 freeze-thaw cycles to lyse the cells. Cell lysate was then heated at 77° C. for 30 min to denature host proteins. After centrifugation at 21,130 g for 5 min, the supernatant was recovered and used as M.TneDI preparations for activity assays. Such prepared M.TneDI can be stored at −20° C. in the presence of 10% (v/v) glycerol for more than 2 months.

### Enzyme Activity Assays of M.TneDI.

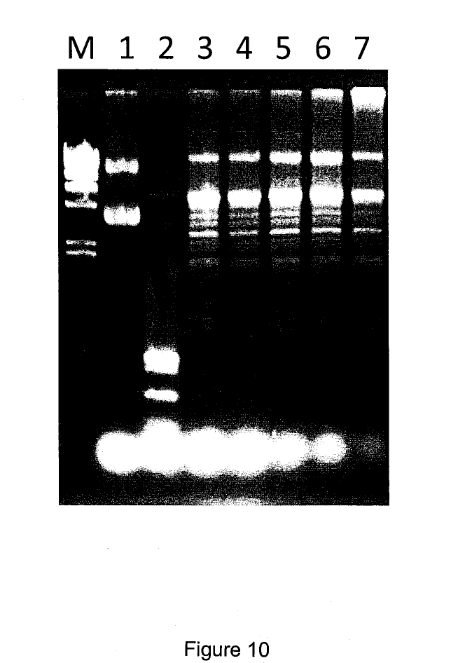

Plasmid pUC19 extracted from a XL1-Blue MRF′ host was used as the substrate DNA (FIG. 10). Eight micrograms of pUC19 was incubated with 10 μl of the above prepared M.TneDI for 1 h at 77° C., supplemented with 20 μg/ml of S-adenosyl-L-methionine (AdoMet). The DNA was then subjected to restriction digestions of R.TneDI or other Type II REases recognizing CGCG sequence, such as BstUI. M.TneDI prevents substrate DNA from the degradation of these nucleases. Based on the growth and the enzyme activity assays, DH10212-3 was identified as the best strain for production of M.TneDI.

Challenges in Cloning and Expressing the TneDI RM System

### Cloning and Expressing R.TneDI.

CTN_{—}0339 encodes the restriction nuclease R.TneDI, which cleaves any DNA carrying CGCG sequences, unless these specific sites have been modified by the cognate methylase M.TneDI. In order to produce large amount of R.TneDI for in vitro applications, it is desirable to clone it to an E. coli strain, such as BL21(DE3), specialized in overexpression of foreign proteins. Cloning restriction nuclease is always a risky attempt, because the chromosomal DNA of the host cell could be degraded by the nuclease. To protect host chromosomal DNA, REases usually have to be cloned in the presence of the cognate MTases (Howard et al. 1986). Alternatively, in vitro transcription and translation approaches could be used (Ishikawa et al. 2005). After considering all of the risks, we decided to impose the expression of R.TneDI under the control of T7 promoter and tried to clone it first in XL1-Blue MRF′. Because XL1-Blue MRF′ does not have T7 RNA polymerase, the transcription of R.TneDI is extremely slow, causing limited damage to the host chromosome. The adaptation of this cloning strategy also took account of the expectation that R.TneDI is likely to be less active at the growth temperatures of E. coli. After R.TneDI had been successfully cloned into XL1-Blue MRF′, we then transferred it (on pJC339) to strain BL21(DE3). Because BL21(DE3) has a T7 RNA polymerase gene, expressing R.TneDI is toxic and potentially fatal to the host, making direct cloning of R.TneDI into BL21(DE3) extremely risky.

### Cloning and Expressing M.TneDI.

CTN_{—}0340 encodes the methylase M.TneDI, which modifies CGCG sequences and protects DNA from the cleavage of R.TneD. Cloning and expressing M.TneDI was complicated by the functions of McrBC genes, which restricts DNA at Pu^{m}C sites, such as G^{m}C. The CGCG sites modified by M.TneDI could be subject to the restriction of McrBC if (1) M.TneDI modifies the second C in the CGCG context, and (2) BL21(DE3) is McrBC positive. Neither of the two scenarios were known when we started the work, so we first have to decide whether BL21(DE3) is McrBC positive. To do so we used a control plasmid pPvuM1.9 (Blumenthal et al., 1985) and a control strain HB101. Plasmid pPvuM1.9 carries M.PvuII, which modifies DNA at G^{m}C sites, and HB101 is a known McrBC negative strain. We attempted to transfer pPvuM1.9 to both HB101 and BL21(DE3). When an equal amount of DNA was used, we were able to get ˜800 times more transformants from HB101 than from BL21(DE3), although the transformability of the two strains differed by only 2 folds. This told us BL21(DE3) is McrBC positive, therefore extra care must been taken when we need to express M.TneDI in this strain. We increased the amount of DNA used for transformation and prepared high quality of competent cells. After several attempts, we finally obtained a BL21(DE3)/pJC340 mutant that is M.TneDI positive but McrBC negative.

III. Genetic Manipulations of Thermotoga Spp.:

### Antibiotics Sensitivity Tests.

One ml of overnight culture was mixed with 25 ml of hot SVO containing 0.3% GELRITE and poured to Petri dishes. Small discs of 7 mm in diameter were cut from Whatman qualitative filter paper and were placed on solidified plates. Various amount of kanamycin (50˜250 μg) was added to the paper discs. After 48 h of anaerobic incubation, sensitive strains would display inhibition zones surrounding the discs. To specify the selective levels of the antibiotic in both liquid and solid media, kanamycin ranging from 50 to 300 μg ml⁻¹ was supplemented, and the proliferation of bacteria was monitored for up to 72 h.

Extraction of DNA from Thermotoga.

Plasmid DNA was extracted from Thermotoga using standard alkaline lysis method. For genomic DNA, overnight culture of Thermotoga was extracted with equal volume of phenol:chloroform:isoamyl alcohol (volume ratio 25:24:1) followed by centrifugation at 13,523 g for 5 min to get rid of cell debris. DNA in the supernatant was precipitated with equal volume of isopropanol, washed once with 70% ethanol, air-dried, and dissolved in 10 mM Tris-EDTA buffer (pH 8.0) containing 20 μg ml⁻¹ RNase.

Construction of pDH10.

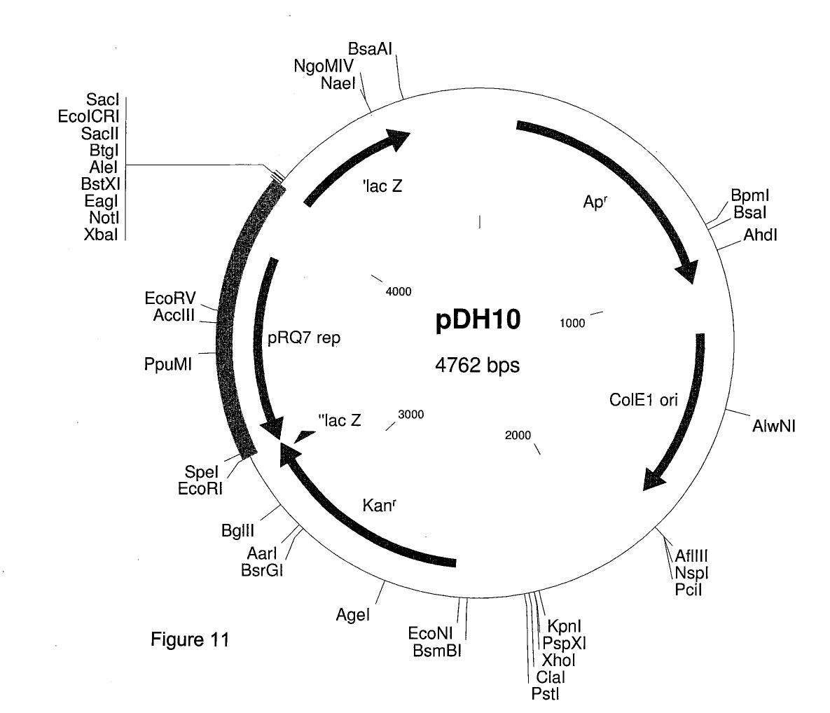

Plasmid pKT1 carrying a thermostable kanamycin adenyl transferase gene (kan) was purchased from Biotools, B & M Labs (Madrid, Spain). Primers 5′-GGGGATCCGAATGTGGTTAGTGTGATTAG-3′ and 5′-GGGAATTCTTAACCATATCCCACTAGTTC-3′ were used to amplify pRQ7, which was linearized immediately after the stop codon of the replication protein as the result of the PCR reactions. The PCR product was digested by BamHI and EcoRI and was inserted into the corresponding sites of pUC19 to give rise to pDH1. The XbaI-EcoRI fragment of pDH1 was then joined to pKT1 pre-digested with the same enzymes to generate pDH10 (FIG. 11). The constructs were confirmed by both restriction digestions and PCR. Plasmid pDH10 carries ColE1 origin of replication (ori) and β-lactamase (Ap^{r}) for amplification and selection in E. coli, respectively. For its replication and selection in Thermotoga, pDH10 relies on the on of pRQ7 and the engineered kan gene. The expression of the kan gene is driven by a promoter from Thermus thermophilus HB8, which is expected to be active in Thermotoga as well, since the consensus sequences of Thermotoga and Thermus promoters are nearly identical.

The invention also contemplates any DNA sequence with at least about 95% sequence identity with pDH10, more preferably at least about 90% sequence identity with pDH10, even more preferably at least about 85% sequence identity with pDH10, still even more preferably at least about 80% sequence identity with pDH10, and most preferably at least about 75% sequence identity with pDH10.

### Transformation and Selection Methods.

Liposome-mediated transformation was conducted as previously described, except that all operations were carried out on the bench top. DOTAP liposomal reagent was purchased from Roche Diagnostics, Indianapolis, Ind., USA. To prepare electrocompetent cells, overnight Thermotoga cultures were transferred to 50 ml of SVO liquid media and were allowed to grow until the cell density reached around 0.2. Cells were collected by centrifugation, washed once with cold deionized water and twice with the washing solution (10% glycerol, 0.85 M sucrose) and were resuspended in 500 μl of the same solution. For electroporation, 4 μg of plasmid DNA was mixed with 50 μl of the freshly made competent cells and incubated on ice for 5 minutes prior to introduction to a pre-chilled cuvette of 1 mm gap. The operation settings were 25 μF capacitance, 200Ω resistance, and 1.5, 1.8, or 2 kV voltage (Gene Pulser Xcell™, Bio-Rad Laboratories, Hercules, Calif.). After electroporation, 1 ml of fresh SVO liquid medium was added to each cuvette, and the cell suspension was transferred to a N₂ serum bottle and incubated at 77° C. with gentle rotation for 3 h for recovery. Half of the recovered culture (500 μl) was then mixed with 25 ml of hot SVO solid medium supplemented with 250 μg ml⁻¹ kanamycin, poured to Petri dishes, and incubated in an anaerobic jar to retrieve transformants.

Stability Assays of the Transformed DNA in Thermotoga and E. coli.

Cultures of Thermotoga recombinant strains were transferred every 12 h for 3 days with an inoculum of 2% to a fresh SVO liquid medium in the presence or absence of kanamycin. With each transfer, an aliquot of the cultures was withdrawn and diluted to 10⁻⁴. Ten microliters of each diluted sample was then mixed with 10 ml of hot SVO solid medium with or without the antibiotic and was poured to a four-section Petri dish. In order to facilitate comparisons, samples of the same strain (but with different treatments) were arranged to different sections of the same plate. After incubation in an anaerobic jar for 48 h, colonies formed in each section were counted and compared.

To test the stability of pDH10 in E. coli, a liquid culture of DH5α/pDH10 was also transferred six times for every 12 h of growth in plain LB medium (no ampicillin) with 1% inoculum. Samples from each cycle were properly diluted and spread on plain LB plates to separate single colonies. One hundred such colonies were randomly chosen and were tested on LB plates containing 100 μg ml⁻¹ ampicillin. For control purposes, strain DH5α/pKT1 was tested in parallel.

Kanamycin is a Suitable Selection Marker for T. sp. RQ7 and T. maritime.

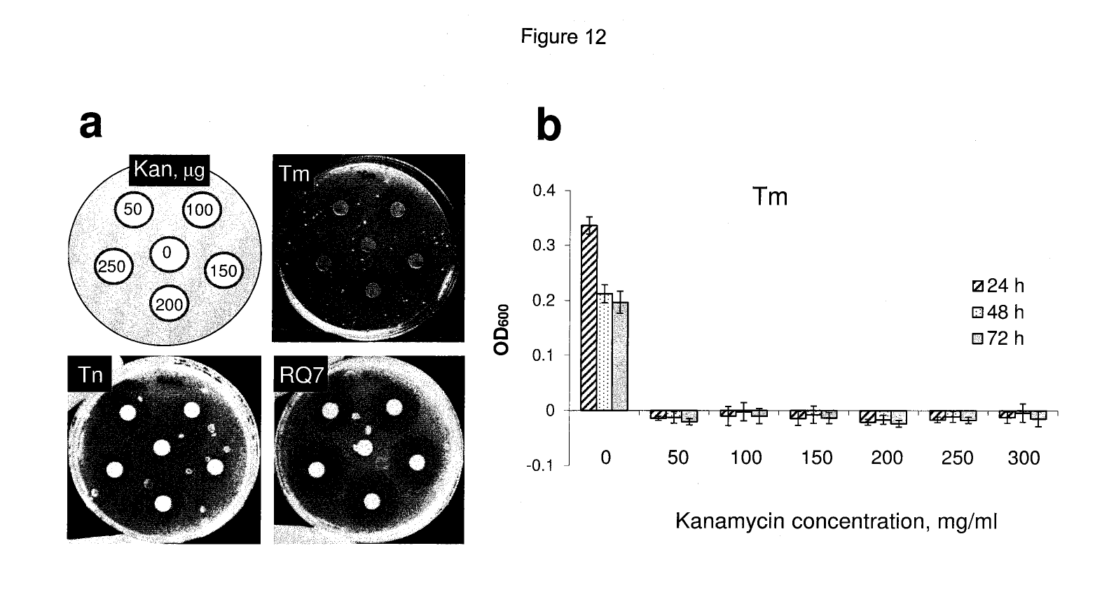

To determine whether kanamycin is a suitable selection marker for Thermotoga, one needs to know the sensitivity of Thermotoga host strains. An initial study indicates that T. maritima is sensitive to kanamycin, but a more recent work states that it is highly resistant to the antibiotic. For T. sp. RQ7, there are simply no related reports. We decided to clarify the discrepancy of the previous findings on T. maritima and to determine the sensitivity of T. sp. RQ7. Small discs of filter paper loaded with various amount of kanamycin were mounted on top of the SVO plates premixed with Thermotoga cultures (FIG. 12a). After two days of incubation, cells not affected by the antibiotic grew into a dense lawn, forming a visible background. Sensitive cells in close proximity to the paper discs were unable to grow, resulting in clear halos. The inhibition zones formed on T. maritima plates (FIG. 12a) revealed that this strain is indeed sensitive to kanamycin. A distinctive zone was visible even with the lowest concentration of kanamycin. T. sp. RQ7 displayed a similar level of sensitivity to the drug. As for T. neapolitana, slight inhibition was noticed when 100 μg of kanamycin was used, and a small inhibition zone was only apparent when 250 μg of the drug was used. Therefore, kanamycin may serve as a good selection marker for T. sp. RQ7 and T. maritima, but not for T. neapolitana.

We next specified the selective levels of kanamycin in both liquid and solid media. The growth of T. maritima in liquid SVO was completely inhibited by 50 μg ml⁻¹ kanamycin for at least 72 h (FIG. 12b). However, spontaneous mutations sometimes caused the cultures to become resistant to the antibiotic, and a complete inhibition over a period of 72 h was only possible when the input amount was increased to 150 μg ml⁻¹. Similar phenomena were also noticed with T. sp. RQ7. On SVO plates, spontaneous mutants of T. maritima and T. sp. RQ7 occasionally appeared after 48 h of incubation when up to 200 μg ml⁻¹ kanamycin was added, but they rarely showed up when the antibiotic concentration was increased to 250 μg ml⁻¹. Based on these observations, for the rest of the study, kanamycin was added at 150 μg ml⁻¹ to liquid media and at 250 μg ml⁻¹ to soft and solid media.

Transformation of Thermotoga-E. coli Shuttle Vector pDH10.

Because most bacteria become competent after a short electric pulse, electroporation was attempted to introduce pDH10 to Thermotoga. Electric pulses of various strengths were applied to both T. sp. RQ7 and T. maritima in the presence of 4 μg of plasmid DNA, and the transformants were selected with embedded growth. When an electric pulse of 2.0 kV was employed, five T. sp. RQ7 and one T. maritima transformants were obtained. A pulse of 1.8 kV resulted in eight T. sp. RQ7 and no T. maritima transformants. When the voltage dropped to 1.5 kV, no transformants were available with either species. These results suggest that the optimal voltage for Thermotoga is around 1.8 to 2.0 kV.

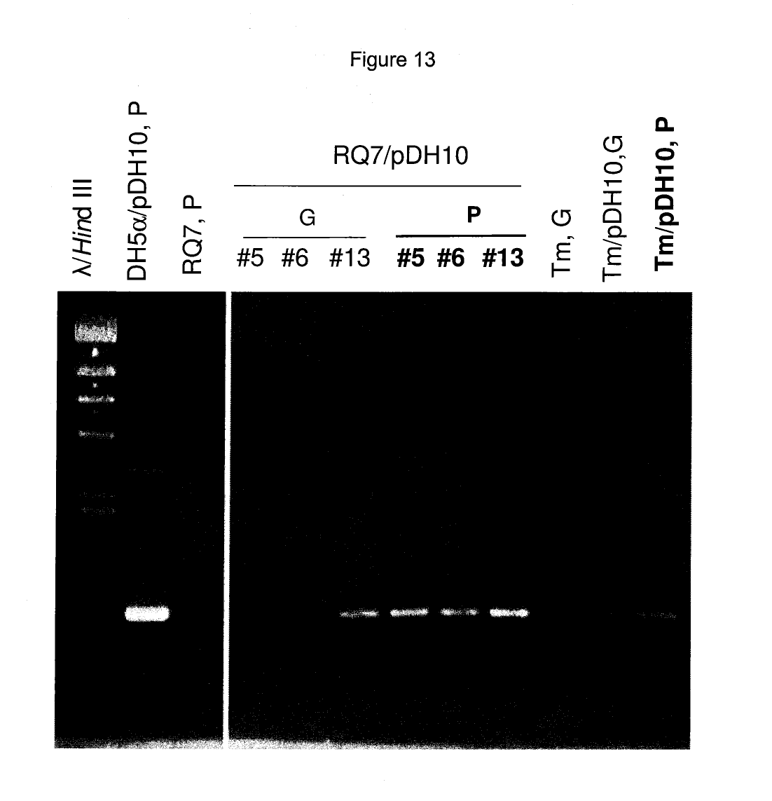

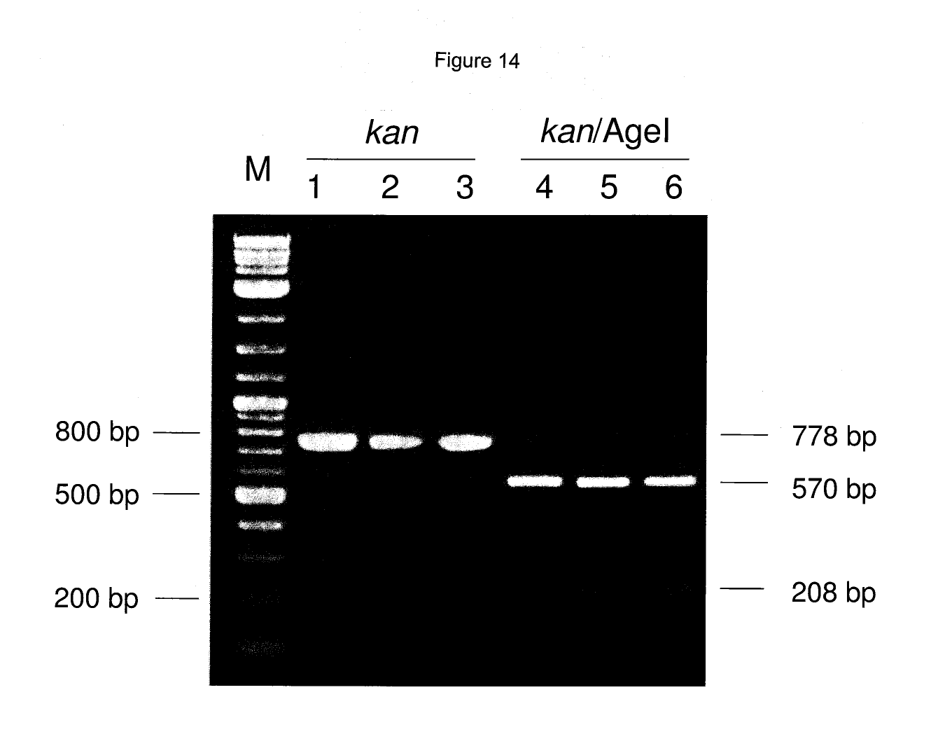

In the control experiment, T. sp. RQ7 and T. maritima cells were treated with a pulse of 1.8 kV in the absence of DNA, and no spontaneous mutants were found. All transformants (designated as RQ7/pDH10 or Tm/pDH10 hereafter) displayed visible growth after a 24 h incubation in soft SVO. Three RQ7/pDH10 strains (#5, #6, and #13) and the single Tm/pDH10 strain were propagated in liquid SVO for extraction of plasmid and genomic DNA. On agarose gels, no pDH10 DNA could be detected from the plasmid extract of any strain, even though pRQ7 was clearly visible from the three RQ7/pDH10 samples, indicating that the extraction procedure was successful. Plasmid and genomic DNA extracted from equal amount of each transformant culture was then subject to PCR analysis. A fragment of 778 bp, corresponding to the size of the kan gene, was obtained from each plasmid extract (lanes are labeled in bold in FIG. 13) but was missing from the genomic DNA of RQ7/pDH10 #5 and #6. The PCR products were gel-purified and were subject to restriction digestion with AgeI, which is expected to cleave the kan gene into two fragments of 208 and 570 bp. Indeed, the digestion reactions released these expected fragments from every sample (FIG. 14), indicating that the PCR products were authentic kan genes.

For validation and comparison purposes, pDH10 was also introduced to T. sp. RQ7 and T. maritima through liposome-mediated transformation. Four T. sp. RQ7 and five T. maritima transformants were obtained from 1 μg of plasmid DNA, as opposed to zero colonies from the samples treated with liposomes containing no DNA. All transformants grew well in both soft and liquid selective media, and the presence of the kan gene was also confirmed by PCR.

### Transformed DNA was Stably Maintained in Thermotoga.

To determine the stability of the transformed DNA, liquid cultures of RQ7/pDH10 and Tm/pDH10 were transferred for every 12 h, for six consecutive times, to fresh media in the presence or absence of kanamycin. Cultures from each transfer cycle were tested with both SVO (no kanamycin) and SVO+Kan plates. The number of colonies on a plain SVO plate represents the quantity of total viable cells in a sample, whereas the number from a SVO+Kan plate defines the abundance of resistant cells. Surprisingly, by the end of the experiment, ˜100% of both RQ7/pDH10 and Tm/pDH10 still had the transformed DNA, even without the selective pressure (Table 3). The kan gene was confirmed by PCR from the plasmid preparations of all strains after each transfer.

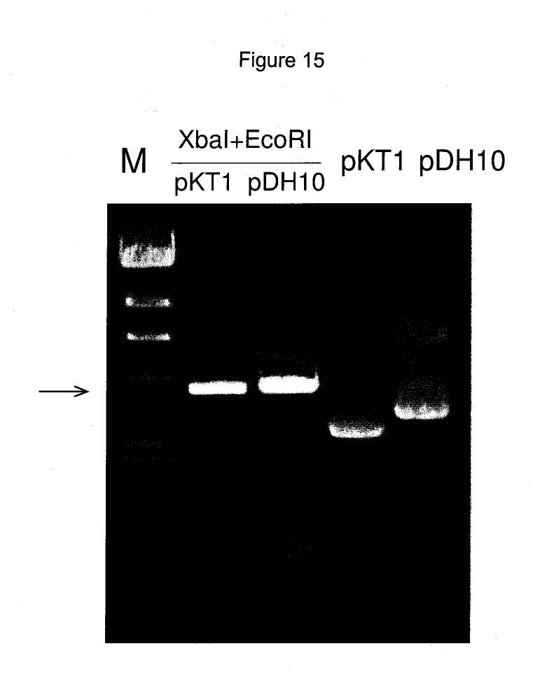

Incorporation of pRQ7 increased the stability of pUC19 derivatives in E. coli. The stable maintenance of the transformed DNA in Thermotoga motivated us to test the stability of pDH10 in E. coli under non-selective conditions. The parent vector pKT1, a derivative of pUC19, was used as the control. Interestingly, pDH10 was much more stable in E. coli than pKT1. The parent vector pKT1 was eliminated by ˜90% of the cells after a single transfer and was completely lost from the population after three transfers (Table 4). By contrast, pDH10 was eliminated at a much slower rate. It was carried by ˜90% of the cells after three transfers and ˜32% of the cells after six transfers. Intact pDH10 was obtained from the resistant colonies. It is noteworthy to mention that similar results were also obtained when pDH1 and pUC19 were compared in the same way. These data demonstrate that the insertion of the pRQ7 sequence somehow enhances the stability of pUC family vectors. We next compared the copy numbers of pDH10 and pKT1 in their E. coli hosts. The two vectors were prepared from the same amount of cells and were digested by XbaI and EcoRI. These double digestions released the pRQ7 sequence from pDH10 (FIGS. 11 and 15). The abundances of the shared vector backbone of pDH10 and pKT1 were comparable on an agarose gel (indicated by the arrow in FIG. 15), suggesting that the copy numbers of two vectors were similar. Therefore, the dramatically improved stability of pDH10 is not caused by an increase in copy number.

### Transformation and Expression of the Kan Gene.

A heterologous kan gene has been functionally expressed and stably established in Thermotoga. The kan gene, carried on the shuttle vector pDH10, was introduced to Thermotoga by both electroporation and liposome-mediated transformation. The latter approach yields higher transformation efficiency (4˜5 transformants per μg of DNA), but the former has the potential to mass-produce competent cells for future needs. Electrocompetency has been established in Thermotoga in this study, and a transformation efficiency of ˜2 transformants per μg of DNA has been observed in RQ7 and ˜0.25 in T. maritima. Two factors might have caused the transformation efficiencies to be low: the damages done by oxygen and the activities of restriction-modification systems. Because the preparation of electrocompetent cells and the transformation were performed under aerobic conditions, a significant portion of the Thermotoga cells would not survive the handling. Having the experiment done in an anaerobic chamber should help to improve the transformation efficiencies, albeit the operations would be cumbersome. In addition, restriction-modification systems have been discovered in Thermotoga, for instance, we have recently characterized a Thermotoga-specific Type II restriction-modification system (details are described above). Unmethylated foreign DNA will be restricted by host-specific restriction nucleases as soon as they enter Thermotoga cells. Proper methylation of pDH10 prior to transformation significantly increases the quantity of transformants. Indeed, electrotransformation efficiency in Clostridia has been improved by 104˜106 folds by methylating the shuttle vectors.

Although intact plasmid DNA of pDH10 has not been detected in Thermotoga, we favor the idea that pDH10 is autonomous in Thermotoga, because the kan gene is always associated to a plasmid preparation instead of a genomic DNA extract (FIG. 13). If pDH10 had been integrated to the chromosome, we would have seen the kan gene showing up more often in genomic DNA samples than in plasmid DNA extracts. Our data stated otherwise (FIG. 13), suggesting that pDH10 is likely an independent molecule rather than part of the chromosome. Because genomic DNA can include both chromosomal and plasmid DNA, it is not surprising to occasionally obtain a positive signal from a genomic DNA sample, even though the kan gene is carried by a free-living plasmid. We suspect that pDH10 has an extremely low copy number in Thermotoga, because the attempts to detect it by inverse PCR (amplifying pDH10 outward from the kan gene), retransformation (transform E. coli with plasmid extracts of Thermotoga transformants), or Southern blotting (using digoxygenin-labeled probes made from either the kan gene or pRQ7) did not generate any signals. The expected inverse PCR product is −4 kb, much bigger than the kan gene. This makes the inverse PCR technically more challenging than just amplifying the kan gene.

### D. References

All references, listed below and elsewhere throughout the description, are hereby incorporated by reference to the extent permitted by applicable law.
Adamczyk-Poplawska M, Lower M, Piekarowicz A (2009) Characterization of the NgoAXP: phase-variable type III restriction-modification system in Neisseria gonorrhoeae. FEMS Microbiology Letters 300(1): 25-35.
Akimkina T, Ivanov P, Kostrov S, Sokolova T, Bonch-Osmolovskaya E, Firman K, Dutta C F, McClellan J A: A highly conserved plasmid from the extreme thermophile Thermotoga maritima MC24 is a member of a family of plasmids distributed worldwide. Plasmid 1999, 42(3):236-240.
Altschul S F, Madden T L, Schaffer A A, Zhang J, Zhang Z, Miller W, Lipman D J (1997) Gapped BLAST and PSI-BLAST: a new generation of protein database search programs. Nucleic Acids Res 25(17): 3389-3402.
Belkin S, Wirsen C O, Jannasch H W (1986) A New Sulfur-Reducing, Extremely Thermophilic Eubacterium from a Submarine Thermal Vent. Applied and Environmental Microbiology 51(6): 1180-1185.
Blumenthal R M, greogry S A, Cooperirder J S (1985) Cloning of a restriction-modification system from Proteus vulgaris and its use in analyzing a methylase-sensitve phenotype in Escherichia coli. J. Baceteriol. 1642:501-509.
Childers S E, Vargas M, Noll K M: Improved Methods for Cultivation of the Extremely Thermophilic Bacterium Thermotoga-Neapolitana. Applied and Environmental Microbiology 1992, 58(12):3949-3953.
Clark J M (1988) Novel non-templated nucleotide addition reactions catalyzed by procaryotic and eucaryotic DNA polymerases. Nucleic Acids Res 16(20): 9677-9686.
Ehrlich M, Gama-Sosa M A, Carreira L H, Ljungdahl L G, Kuo K C, Gehrke C W (1985) DNA methylation in thermophilic bacteria: N4-methylcytosine, 5-methylcytosine, and N6-methyladenine. Nucleic Acids Res 13(4): 1399-1412.
Gaido M L, Prostko C R, Strobl J S (1988) Isolation and characterization of BsuE methyltransferase, a CGCG specific DNA methyltransferase from Bacillus subtilis. J Biol Chem 263(10): 4832-4836.
Gaido M L, Strobl J S (1987) Methylation sensitivity of the restriction enzymes FnuDII and AccII. Arch Microbiol 146(4): 338-340.
Gong W, O'Gara M, Blumenthal R M, Cheng X (1997) Structure of pvu II DNA-(cytosine N4) methyltransferase, an example of domain permutation and protein fold assignment. Nucleic Acids Res 25(14): 2702-2715.
Grant S G, Jessee J, Bloom F R, Hanahan D: Differential plasmid rescue from transgenic mouse DNAs into Escherichia coli methylation-restriction mutants. Proceedings of the National Academy of Sciences of the United States of America 1990, 87(12):4645-4649.
Han, Dongmei, Norris, Stephen M., Xu, Zhaohui (2012) Construction and transformatin of a Thermotoga-E. coli shuttle vector. BMC Biotechnology 12:2.
Harriott O T, Huber R, Stetter K O, Betts P W, Noll K M: A Cryptic Miniplasmid from the Hyperthermophilic Bacterium Thermotoga Sp Strain Rq7. Journal of bacteriology 1994, 176(9):2759-2762.
Hermann M, Noll K M, Wolfe R S: Improved Agar Bottle Plate for Isolation of Methanogens or Other Anaerobes in a Defined Gas Atmosphere. Appl Environ Microbiol 1986, 51(5):1124-1126.
Hjorleifsdottir S, Petursdottir S K, Korpela J, Torsti A M, Mattila P, Kristjansson J K (1996) Screening for restriction endonucleases in aerobic, thermophilic eubacteria. Biotechnology Techniques 10(1): 13-18.
Howard K A, Card C, Benner J S, Callahan H L, Maunus R, Silber K, Wilson G, Brooks J E (1986) Cloning the Ddel restriction-modification system using a two-step method. Nucleic Acids Res 14(20): 7939-7951.
Huber R, Langworthy T A, Konig H, Thomm M, Woese C R, Sleytr U B, Stetter K O: Thermotoga maritima sp. nov. represents a new genus of unique extremely thermophilic eubacteria growing up to 90 degrees C. Archives of Microbiology 1986, 144(4):324-333.
Ishikawa K, Watanabe M, Kuroita T, Uchiyama I, Bujnicki J M, Kawakami B, Tanokura M, Kobayashi I (2005) Discovery of a novel restriction endonuclease by genome comparison and application of a wheat-germ-based cell-free translation assay: Pabl (5′-GTA/C) from the hyperthermophilic archaeon Pyrococcus abyssi. Nucleic Acids Res 33(13): e112.
Jiang Y, Zhou Q, Wu K, Li X Q, Shao W L: A highly efficient method for liquid and solid cultivation of the anaerobic hyperthermophilic eubacterium Thermotoga maritima. Fems Microbiology Letters 2006, 259(2):254-259.
Lasa I, Caston J R, Fernandez-Herrero L A, de Pedro M A, Berenguer J: Insertional mutagenesis in the extreme thermophilic eubacteria Thermus thermophilus HB8. Molecular microbiology 1992, 6(11):1555-1564.
Le Fourn C, Fardeau M L, Ollivier B, Lojou E, Dolla A: The hyperthermophilic anaerobe Thermotoga Maritima is able to cope with limited amount of oxygen: insights into its defence strategies. Environmental microbiology 2008, 10(7):1877-1887.
Liao D, Dennis P P: The organization and expression of essential transcription translation component genes in the extremely thermophilic eubacterium Thermotoga maritima. The Journal of biological chemistry 1992, 267(32):22787-22797.
Liao H, McKenzie T, Hageman R: Isolation of a thermostable enzyme variant by cloning and selection in a thermophile. Proceedings of the National Academy of Sciences of the United States of America 1986, 83(3):576-580.
Lui A C, McBride B C, Vovis G F, Smith M (1979) Site specific endonuclease from Fusobacterium nucleatum. Nucleic Acids Res 6(1): 1-15.
Macy J M, Snellen J E, Hungate R E: Use of syringe methods for anaerobiosis. The American journal of clinical nutrition 1972, 25(12):1318-1323.
Malone T, Blumenthal R M, Cheng X (1995) Structure-guided analysis reveals nine sequence motifs conserved among DNA amino-methyltransferases, and suggests a catalytic mechanism for these enzymes. J Mol Biol 253(4): 618-632.
Maseda H, Hoshino T: Screening and analysis of DNA fragments that show promoter activities in Thermus thermophilus. FEMS Microbiol Lett 1995, 128(2):127-134.
Matveyev A V, Young K T, Meng A, Elhai J (2001) DNA methyltransferases of the cyanobacterium Anabaena PCC 7120. Nucleic Acids Res 29(7): 1491-1506.
Mermelstein L D, Papoutsakis E T: In vivo methylation in Escherichia coli by the Bacillus subtilis phage phi 3T I methyltransferase to protect plasmids from restriction upon transformation of Clostridium acetobutylicum ATCC 824. Appl Environ Microbiol 1993, 59(4):1077-1081.
Miller T L, Wolin M J: A serum bottle modification of the Hungate technique for cultivating obligate anaerobes. Applied microbiology 1974, 27(5):985-987.
Miyake M, Kotani H, Asada Y (1992) Isolation and identification of restriction endonuclease, Sell from a cyanobacterium, Synechococcus elongatus. Nucleic Acids Res 20(10): 2605.
Morana A, Stiuso P, Colonna G, Lamberti M, Carteni M, De Rosa M (2002) Stabilization of S-adenosyl-L-methionine promoted by trehalose. Biochim Biophys Acta 1573(2): 105-108.
Nesbo C L, Dlutek M, Doolittle W F: Recombination in thermotoga: Implications for species concepts and biogeography. Genetics 2006, 172(2):759-769.
Pingoud A, Fuxreiter M, Pingoud V, Wende W (2005) Type II restriction endonucleases: structure and mechanism. Cell Mol Life Sci 62(6): 685-707.
Roberts R J, Belfort M, Bestor T, Bhagwat A S, Bickle T A, Bitinaite J, Blumenthal R M, Degtyarev S, Dryden D T, Dybvig K, Firman K, Gromova E S, Gumport R I, Halford S E, Hattman S, Heitman J, Hornby D P, Janulaitis A, Jeltsch A, Josephsen J, Kiss A, Klaenhammer T R, Kobayashi I, Kong H, Kruger D H, Lacks S, Marinus M G, Miyahara M, Morgan R D, Murray N E, Nagaraja V, Piekarowicz A, Pingoud A, Raleigh E, Rao D N, Reich N, Repin V E, Selker E U, Shaw P C, Stein D C, Stoddard B L, Szybalski W, Trautner T A, Van Etten J L, Vitor J M, Wilson G G, Xu S Y (2003) A nomenclature for restriction enzymes, DNA methyltransferases, homing endonucleases and their genes. Nucleic Acids Res 31(7): 1805-1812.
Roberts R J, Vincze T, Posfai J, Macelis D (2010) REBASE—a database for DNA restriction and modification: enzymes, genes and genomes. Nucleic Acids Res 38(Database issue): D234-236.
Sambrook J, Russell D W: The condensed protocols from molecular cloning: a laboratory manual: Cold Spring Harbor Laboratory Press; 2006.
Schroder C, Selig M, Schonheit P (1994) Glucose Fermentation to Acetate, CO2 and H2 in the Anaerobic Hyperthermophilic Eubacterium Thermotoga Maritima: Involvement of the Embden-Meyerhof Pathway. Archives of Microbiology 161(6): 460-470.
Sears A, Peakman L J, Wilson G G, Szczelkun M D (2005) Characterization of the Type III restriction endonuclease PstII from Providencia stuartii. Nucleic Acids Res 33(15): 4775-4787.
Slatko B E, Benner J S, Jager-Quinton T, Moran L S, Simcox T G, Van Cott E M, Wilson G G (1987) Cloning, sequencing and expression of the Taq I restriction-modification system. Nucleic Acids Res 15(23): 9781-9796.
Stier I, Kiss A (2010) The type II restriction endonuclease MvaI has dual specificity. Nucleic Acids Res 38(22): 8231-8238.
Strobl J S, Thompson E B (1984) Methylation of either cytosine in the recognition sequence CGCG inhibits ThaI cleavage of DNA. Nucleic Acids Res 12(21): 8073-8083.
Studier F W, Rosenberg A H, Dunn J J, Dubendorff J W (1990) Use of T7 RNA polymerase to direct expression of cloned genes. Methods Enzymol 185: 60-89.
Takahata Y, Nishijima M, Hoaki T, Maruyama T: Thermotoga petrophila sp. nov. and Thermotoga naphthophila sp. nov., two hyperthermophilic bacteria from the Kubiki oil reservoir in Niigata, Japan. International Journal of Systematic and Evolutionary Microbiology 2001, 51:1901-1909.
Thomm M, Frey G, Bolton B J, Laue F, Kessler C, Stetter K O (1988) MvnI: A restriction enzyme in the archaebacterium Methanococcus vannielii. FEMS Microbiology Letters 52(3): 229-233.
Thompson J D, Higgins D G, Gibson T J (1994) CLUSTAL W: improving the sensitivity of progressive multiple sequence alignment through sequence weighting, position-specific gap penalties and weight matrix choice. Nucleic Acids Res 22(22): 4673-4680.
Yu J S, Noll K M: Plasmid pRQ7 from the hyperthermophilic bacterium Thermotoga species strain RQ7 replicates by the rolling-circle mechanism. Journal of bacteriology 1997, 179(22):7161-7164.
Yu J S, Vargas M, Mityas C, Noll K M: Liposome-mediated DNA uptake and transient expression in Thermotoga. Extremophiles 2001, 5(1):53-60.
Van Ooteghem S A, Beer S K, Yue P C (2002) Hydrogen production by the thermophilic bacterium Thermotoga neapolitana. Applied Biochemistry and Biotechnology 98: 177-189.
Van Ooteghem S A, Jones A, Van Der Lelie D, Dong B, Mahajan D: H(2) production and carbon utilization by Thermotoga neapolitana under anaerobic and microaerobic growth conditions. Biotechnol Lett 2004, 26(15):1223-1232.
Venetianer P, Orosz A (1988) BepI restriction endonuclease, a new isoschisomer of FnuDII. Nucleic Acids Res 16(1): 350.
Whitehead P R, Brown N L (1985) A simple and rapid method for screening bacteria for type II restriction endonucleases: enzymes in Aphanothece halophytica. Arch Microbiol 141(1): 70-74.
Wilson G G, Murray N E (1991) Restriction and modification systems. Annu Rev Genet. 25: 585-627.
Windberger E, Huber R, Trincone A, Fricke H, Stetter K O: Thermotoga thermarum sp. nov. and Thermotoga neapolitana occurring in African continental solfataric springs. Archives of Microbiology 1989, 151(6):506-512.
Xu, Zhaohui, Han, Dongmei, Cao, Jingjing, Saini, Uksha (2011) Cloning and characterization of the TneDI restriction-modification system of Thermotoga neapolitana. Extremophiles 15:665-672; DOI 10.1007/s00792-011-0397-9.

### E. Tables

TABLE 1
Strains & vectors

| Strain or | | |
|---|---|---|
| plasmid | Description | Reference |
| Thermotoga | | |
| T. neapolitana | Isolated from a shallow submarine hot spring | (Belkin et al. 1986) |
| ATCC 49049 | near Bay of Naples, Italy | |
| T. maritima | Isolated from geothermically heated sea floors in | Huber et al. (1986) |
| MSB8 | Italy and the Azores | |
| T. sp. RQ7 | Isolated from geothermically heated sea floors in | Nesbo et al. (2006) |
| | Ribeira Quente, the Azores | |
| E. coli | | |
| DH5α | F′ endA1 hsdR17 (rₖ−, mₖ+) supE44 thi-1 λ- | Grant et al. (1990) |
| | recA1 gyrA96 relA1 deoR Δ(lacZYA-argF)-U169 | |
| | φ80dlacZΔM15 | |
| XL1-Blue MRF′ | Δ(mcrA)183 Δ(mcrCB-hsdSMR-mrr)173 endA1 | Strategene |
| | supE44 thi-1 recA1 gyrA96 relA1 lac [F′ proAB | |
| | lacI^{q}ZΔM15 Tn10 (Tet^{r})] | |
| BL21(DE3) | F⁻ ompT gal [dcm] [lon] hsdS_{B} (r_{B}⁻ m_{B}⁻) with DE3, | (Studier et al. 1990) |
| | a λ prophage carrying the T7 RNA polymerase | |
| | gene | |
| Plasmids | | |
| pUC19 | High-copy number E. coli cloning vector | GenBank Accession |
| | containing portions of pBR322 and M13mp19; | #: L09137 |
| | Ap^{r} | |
| pET-24a(+) | E. coli expression vector possessing an N- | Novagen |
| | terminal T7·Tag ® sequence and a C-terminal | |
| | His·Tag ® sequence; Kn^{r} | |
| pACYC184 | Low-copy number E. coli cloning vector | GenBank Accession |
| | containing the p15A origin of replication; Tc^{r}; Cm^{r} | #: X06403 |
| pJC184 | A pACYC184-derived plasmid with a NdeI- | Xu et al. (2011) |
| | BamHI linker inserted downstream the promoter | |
| | of tet; Cm^{r} | |
| pJC339 | Coding region of CTN_0339 (excluding the stop | Xu et al. (2011) |
| | codon) inserted into the NdeI-HindIII sites of | |
| | pET-24a(+); Kn^{r} | |
| pJC340 | Coding region of CTN_0340 inserted into the | Xu et al. (2011) |
| | NdeI-BamHI sites of pJC184; Cm^{r} | |
| pDH22 | Coding region of CTN_0339 was digested by | Xu et al. (2011) |
| | NdeI and R.TneDI and was inserted into the | |
| | NdeI-SmaI sites of pUC19; Ap^{r} | |
| pRQ7 | 846 bp cryptic miniplasmid from T. sp. RQ7 | Harriott et al. (1994) |
| pKT1 | 3934 bp pUC-derived plasmid, containing a kan | Lasa et al. (1992) |
| | cassette for thermostable kanamycin selections | |
| pDH1 | 3517 bp pRQ7 DNA cloned between BamH1 and | Han et al. (2012) |
| | EcoR1 sites of pUC19; Ap^{r} | |
| pDH10 | 4792 bp pRQ7 DNA cloned between EcoR1 and | Han et al. (2012); |
| | Xba1 sites of pKT1; Ap^{r}; Kan^{r} | GenBank: |
| | | JN813374 |
| pDH21 | Coding region of CTN_0340 inserted into the | this disclosure |
| | NdeI and BamHI sites of pET-24a(+) | |

TABLE 2
Orthologs of the TneDI system*

| | R.TneDI | M.TneDI |
|---|---|---|
| T. maritima | TM0329 (100) | TM0328 (100) |
| T. petrophila RKU-1 | Tpet_0590 (100) | Tpet_0591 (100) |
| T. sp. RQ2 | TRQ2_0604 (100) | TRQ2_0605 (100) |
| Marinitoga piezophila | MPKA3_1285 (100) | MPKA3_1161 (100) |
| KA3 | | |
| T. naphthophila RKU-10 | Tnap_0965 (93) | Tnap_0964 (98) |
| F. placidus | Ferp_1585 (38) | Ferp_1586 (76) |

*Identities of the protein products pertaining to TneDI system are listed as percentages in parentheses as aligned by BLASTP (Altschul et al. 1997).

TABLE 3
Percentage of Thermotoga colonies resistant to kanamycin after six
transfers**

| Recombinant | | |
|---|---|---|
| Strain | Transfer Medium | Resistant colonies (%) |
| RQ7/pDH10 | SVO | 104.92 ± 9.57 |
| | SVO + Kan | 106.66 ± 3.56 |
| Tm/pDH10 | SVO | 100.96 ± 23.72 |
| | SVO + Kan | 108.59 ± 7.54 |

**Results of three independent tests

TABLE 4
Percentage of E. coli colonies resistant to ampicillin during consecutive
transfers***

| Number of transfers | pDH10 | pKT1 |
|---|---|---|
| 1 | 96.75 ± 1.30 | 7.25 ± 5.54 |
| 2 | 95.5 ± 2.50 | 1.75 ± 1.48 |
| 3 | 90 ± 6.04 | 0 |
| 4 | 68.5 ± 6.34 | — |
| 5 | 44 ± 11.68 | — |
| 6 | 32.25 ± 15.20 | — |

***Results of four independent tests

In accordance with the provisions of the patent statutes, the present invention has been described in what is considered to represent its preferred embodiments. However, it should be noted that the invention can be practiced otherwise than as specifically illustrated and described without departing from its spirit or scope.

What is claimed:

1. A method of preparing solid cultures of anaerobic microbes comprising the steps of:
(a) heating an amount of liquid media;
(b) heating an amount of solidifying agent;
(c) mixing equal volumes of said amount of liquid media and said amount of solidifying agent;
(d) pouring said equal volumes of said amount of liquid media and said amount of solidifying agent into a first vessel;
(e) allowing said equal volumes of said amount of liquid media and said amount of solidifying agent to solidify in first vessel, thereby creating a solid media,
(e) placing said first vessel containing said solid media within a second vessel capable of sustaining anaerobic cultivation; and
(f) incubating said vessels.

2. A method of transferring, under aerobic conditions, a colony of anaerobic microbes from solid media to liquid media comprising the steps of:
(a) creating soft media by adding an amount of solidifying agent to an amount of liquid media, wherein the amount of solidifying agent added is less than an amount of solidifying agent in the solid media, and also wherein the amount of solidifying agent added is more than an amount of solidifying agent in the liquid media;
(b) removing said colony from said solid media;
(c) placing said colony within the soft media, thereby creating an inoculated soft media;
(e) incubating said inoculated soft media for an incubation period;
(f) after said incubation period, transferring an inoculum of anaerobic microbes from the inoculated soft media to liquid media.

3. The method of claim 2 wherein said solid, soft, and liquid media each comprise a reducing agent.

4. The method of claim 2 wherein said solid, soft, and liquid media each comprise SVO media.

5. The method of claim 2 wherein said anaerobic microbes comprise microbes of Thermotoga spp.

6. The method of claim 2 wherein said colony is placed under a thickness of soft media.

7. The method of claim 1 wherein said amount of liquid media comprises anaerobic microbes.

8. An isolated or recombinant DNA sequence coding for R.TneDI or a functional derivative thereof.

9. A vector comprising a DNA sequence according to claim 8.

10. A host cell transformed with the vector of claim 9.

11. A process for the manufacture of R.TneDI or a functional derivative thereof, comprising cultivation of cells transformed with a recombinant vector according to claim 9 in the nutrient medium allowing expression of said R.TneDI or functional derivative thereof.

12. R.TneDI or a functional derivative thereof whenever prepared by the process of claim 11.

13. An isolated or recombinant DNA sequence coding for M.TneDI or a functional derivative thereof.

14. A vector comprising a DNA sequence according to claim 13.

15. A host cell transformed with the vector of claim 14.

16. A process for the manufacture of M.TneDI or a functional derivative thereof, comprising cultivation of cells transformed with a recombinant vector according to claim 14 in the nutrient medium allowing expression of said M.TneDI or functional derivative thereof.

17. M.TneDI or a functional derivative thereof whenever prepared by the process of claim 16.

18. A vector capable of replication in both Thermatoga and non-Thermotoga species.

19. A vector according to claim 18 having at least about 75% homology to pDH10.


SEQUENCE LISTING (23 sequences)

SEQ ID NO: 1 - DNA, 2242 bp, Artificial Sequence
  plasmid
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgcatatggaattcccgcggatcctgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 2 - DNA, 2949 bp, Artificial Sequence
  plasmid
tatgagaaaaacggatcctctcattgatattttcaaaaagaacaaagagagaatcgatatccttggaaaagcatgggatatcttttattttgaaggaagagctcccgatgtaggtaaaaggcgtgagaatttcatagttgagatgataagaaaagaactttcacacttcataaaatccgttaatcaagccccagacacagaaagaaactgggatattgaaataacttttcaagacgactcaacaaagaggtacagtataaaaacaactgaaggattttccgacgttaaaatagcatgggatggattcccaaccaaagatagaattttaactttcaacttccaggcagatatattctacattgcaagaaacaaagacaaaataatactatgcgttattgatttagaaaaattgagagacctacagagagaagtagaaaaaaatgaatcaaagttggaggagtactactcactcccgagcagcaacacaaacccgggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacca

SEQ ID NO: 3 - DNA, 33 bp, Artificial Sequence
  primer
ggaattccatatgagaaaaacggatcctctcat

SEQ ID NO: 4 - DNA, 29 bp, Artificial Sequence
  primer
cacaagcttctgttgatatttttctatca

SEQ ID NO: 5 - DNA, 32 bp, Artificial Sequence
  primer
gaattccatatgcggtgcctgactgcgttagc

SEQ ID NO: 6 - DNA, 55 bp, Artificial Sequence
  primer
gaattccatatggaattcccgcggatcctgagaagcacacggtcacactgcttcc

SEQ ID NO: 7 - DNA, 32 bp, Artificial Sequence
  primer
gaattccatatgaagagaagaaaatcaacaag

SEQ ID NO: 8 - DNA, 29 bp, Artificial Sequence
  primer
cgcggatccttatcaacagtgatcttcaa

SEQ ID NO: 9 - DNA, 5921 bp, Artificial Sequence
  plasmid
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttctgttgatatttttctatcaactcttttatgagatcgtaccatcctttgaaatattcttcttttgctttgagtatcaattcaacgggaaaaggtttgtaatcgatctctaaatagttttcctctttttttgaaagctcgatcaatctttttacagtagaactcttcaatccaaaaccacgcgggtttgtgttgctgctcgggagtgagtagtactcctccaactttgattcatttttttctacttctctctgtaggtctctcaatttttctaaatcaataacgcatagtattattttgtctttgtttcttgcaatgtagaatatatctgcctggaagttgaaagttaaaattctatctttggttgggaatccatcccatgctattttaacgtcggaaaatccttcagttgtttttatactgtacctctttgttgagtcgtcttgaaaagttatttcaatatcccagtttctttctgtgtctggggcttgattaacggattttatgaagtgtgaaagttcttttcttatcatctcaactatgaaattctcacgccttttacctacatcgggagctcttccttcaaaataaaagatatcccatgcttttccaaggatatcgattctctctttgttctttttgaaaatatcaatgagaggatccgtttttctcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 10 - DNA, 3159 bp, Artificial Sequence
  plasmid
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgcatatgaagagaagaaaatcaacaagaaccagttcttttggagtaaaaggccgggaaagccatgattcatctccattttatagtagagcgatctattccaacttttcccttccaaaaccttcagaggaagatctcatcgagaacccacttcctgaaaatctcttagacaaggtcattgagggggatgccagagaagttctgaaaaaaatacctgactgttcgatacatctcatggtgacttcacctccgtacaatgtagggaaagaatacgatgaagatatgactcttgatgaataccttgaattcatagaagaaatcatgaaagaagtgtacagggttctcgtctggggtggaagagcctgttttaatgtagcaaatcttggaagaaaaccgtacattcctcttcatgcgtatctcatccatttgtttgaaaagataggctttttgatacgtggagagatcatttgggacaaaggggaagcggtgagtggttcttcgactgcgtggggttcgtggatgtctcctgtaaatcccgtgctaagagatcaacacgaatatataattgtaatgagtaaaggagatctaaagcgaagaaaacccagtgatagagaagcaaaatcaacaataacaagggaagaatttttagaattcacaaggagtgtgtggaaatttccaccagagtcagctaagagagtaggacatcctgccccctttcctgaggaacttccttaccgttgtatacagctctacacctttaaaggagatgtcgttctcgatccttttgctggtgtaggaacaacttgtgtggctgccgttaaaacaggacggcattttgttggaatagagataaatcccgaatacgtaaaaaaggccgaagaaagggtaaaggatattctcagcaaacctactctttttgaagatcactgttgataaggatcctgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 11 - DNA, 672 bp, Thermotoga neapolitana
atgagaaaaacggatcctctcattgatattttcaaaaagaacaaagagagaatcgatatccttggaaaagcatgggatatcttttattttgaaggaagagctcccgatgtaggtaaaaggcgtgagaatttcatagttgagatgataagaaaagaactttcacacttcataaaatccgttaatcaagccccagacacagaaagaaactgggatattgaaataacttttcaagacgactcaacaaagaggtacagtataaaaacaactgaaggattttccgacgttaaaatagcatgggatggattcccaaccaaagatagaattttaactttcaacttccaggcagatatattctacattgcaagaaacaaagacaaaataatactatgcgttattgatttagaaaaattgagagacctacagagagaagtagaaaaaaatgaatcaaagttggaggagtactactcactcccgagcagcaacacaaacccgcgtggttttggattgaagagttctactgtaaaaagattgatcgagctttcaaaaaaagaggaaaactatttagagatcgattacaaaccttttcccgttgaattgatactcaaagcaaaagaagaatatttcaaaggatggtacgatctcataaaagagttgatagaaaaatatcaacag

SEQ ID NO: 12 - DNA, 927 bp, Thermotoga neapolitana
atgaagagaagaaaatcaacaagaaccagttcttttggagtaaaaggccgggaaagccatgattcatctccattttatagtagagcgatctattccaacttttcccttccaaaaccttcagaggaagatctcatcgagaacccacttcctgaaaatctcttagacaaggtcattgagggggatgccagagaagttctgaaaaaaatacctgactgttcgatacatctcatggtgacttcacctccgtacaatgtagggaaagaatacgatgaagatatgactcttgatgaataccttgaattcatagaagaaatcatgaaagaagtgtacagggttctcgtctggggtggaagagcctgttttaatgtagcaaatcttggaagaaaaccgtacattcctcttcatgcgtatctcatccatttgtttgaaaagataggctttttgatacgtggagagatcatttgggacaaaggggaagcggtgagtggttcttcgactgcgtggggttcgtggatgtctcctgtaaatcccgtgctaagagatcaacacgaatatataattgtaatgagtaaaggagatctaaagcgaagaaaacccagtgatagagaagcaaaatcaacaataacaagggaagaatttttagaattcacaaggagtgtgtggaaatttccaccagagtcagctaagagagtaggacatcctgccccctttcctgaggaacttccttaccgttgtatacagctctacacctttaaaggagatgtcgttctcgatccttttgctggtgtaggaacaacttgtgtggctgccgttaaaacaggacggcattttgttggaatagagataaatcccgaatacgtaaaaaaggccgaagaaagggtaaaggatattctcagcaaacctactctttttgaagatcactgttga

SEQ ID NO: 13 - protein, 224 aa, Thermotoga neapolitana
MRKTDPLIDIFKKNKERIDILGKAWDIFYFEGRAPDVGKRRENFIVEMIRKELSHFIKSVNQAPDTERNWDIEITFQDDSTKRYSIKTTEGFSDVKIAWDGFPTKDRILTFNFQADIFYIARNKDKIILCVIDLEKLRDLQREVEKNESKLEEYYSLPSSNTNPRGFGLKSSTVKRLIELSKKEENYLEIDYKPFPVELILKAKEEYFKGWYDLIKELIEKYQQ

SEQ ID NO: 14 - protein, 308 aa, Thermotoga neapolitana
MKRRKSTRTSSFGVKGRESHDSSPFYSRAIYSNFSLPKPSEEDLIENPLPENLLDKVIEGDAREVLKKIPDCSIHLMVTSPPYNVGKEYDEDMTLDEYLEFIEEIMKEVYRVLVWGGRACFNVANLGRKPYIPLHAYLIHLFEKIGFLIRGEIIWDKGEAVSGSSTAWGSWMSPVNPVLRDQHEYIIVMSKGDLKRRKPSDREAKSTITREEFLEFTRSVWKFPPESAKRVGHPAPFPEELPYRCIQLYTFKGDVVLDPFAGVGTTCVAAVKTGRHFVGIEINPEYVKKAEERVKDILSKPTLFEDHC

SEQ ID NO: 15 - DNA, 12 bp, Artificial Sequence
  ligation site
tccaaaaccacg

SEQ ID NO: 16 - DNA, 18 bp, Artificial Sequence
  ligation site
cacaaacccgggggatcc

SEQ ID NO: 17 - protein, 336 aa, Proteus hauseri
MMTLNLQTMSSNDMLNFGKKPAYTTSNGSMYIGDSLELLESFPDESISLVMTSPPFALQRKKEYGNLEQHEYVDWFLSFAKVVNKKLKPDGSFVVDFGGAYMKGVPARSIYNFRVLIRMIDEVGFFLAEDFYWFNPSKLPSPIEWVNKRKIRVKDAVNTVWWFSKTEWPKSDITKVLAPYSDRMKKLIEDPDKFYTPKTRPSGHDIGKSFSKDNGGSIPPNLLQISNSESNGQYLANCKLMGIKAHPARFPAKLPEFFIRMLTEPDDLVVDIFGGSNTTGLVAERESRKWISFEMKPEYVAASAFRFLDNNISEEKITDIYNRILNGESLDLNSII

SEQ ID NO: 18 - protein, 5 aa, Thermotoga neapolitana
  MISC_FEATURE (5)..(5): Xaa represents Phe or Tyr
TSPPX

SEQ ID NO: 19 - protein, 5 aa, Thermotoga neapolitana
  MISC_FEATURE (2)..(2): Xaa represents any amino acid
FXGXX

SEQ ID NO: 20 - DNA, 4762 bp, Artificial Sequence
  plasmid
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctgggtaccgggccccccctcgaggtcgacggtatcgataagcttggctgcaggtcgacggatccccgggagtataacagaaaccttaaggcccgaccgcttgacaagggcgcgtgaggtttttacgatagcgccggatgcggggaaaaagggctccttttggggggttttccccgcaccgggcggacctgggcgcagaggaaacgcggcaactcgcccgtctcgggttcccgcccacgacccttaaggaggtgtgaggcatatgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaatttttatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaaagttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcgctcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacggaattcttaaccatatcccactagttcccaaattcttccccaccttataccctctggtggtatctcaataatcaacttctcccatattttgataatctctttcatctcgtaccttaatgctctacataaccacttccacacttgcgcagcccctttgaaaatccacttccaactatacccgaatcttccttccatctcctttgataagtacttcatcatatatcgtattgctttacctgtgtcccatactgcttctacataaacgatttttgccttatgaatctcttcccaattcttacttatccaatcttggggaatataaggtcctctaaataatatatgcatatgcaccaaaccactttgagtacgttcccgtacccaaatgtattcaaaattaccatatcttctccggattcgttttttaagcttttcccagctatgtgtgatatcggcaggagattccaatgaagaggtaagcgtcaggaaggctataaattcgttcctatgcttaccagcagttacaccacttacagccctctggtagaatctcttttttcgccttgtagacgccagcggcgacttatgacatttactatcaagtgaaccttgctcgaccctattttgtgttataatctcttttgacatcgggtagttacaccgcctttcttttggatttttccccgggtgtatgtgggtacacccggggttgtttcattattatgagccctactaaatattataagcaccatgtgttccctataggtcacaccataaccttatcttaatccttatcatcttaagccttatcataatccttatcgtaacccttatctaatcacactaaccacattcggatcctctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 21 - DNA, 6204 bp, Artificial Sequence
  plasmid
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccttatcaacagtgatcttcaaaaagagtaggtttgctgagaatatcctttaccctttcttcggccttttttacgtattcgggatttatctctattccaacaaaatgccgtcctgttttaacggcagccacacaagttgttcctacaccagcaaaaggatcgagaacgacatctcctttaaaggtgtagagctgtatacaacggtaaggaagttcctcaggaaagggggcaggatgtcctactctcttagctgactctggtggaaatttccacacactccttgtgaattctaaaaattcttcccttgttattgttgattttgcttctctatcactgggttttcttcgctttagatctcctttactcattacaattatatattcgtgttgatctcttagcacgggatttacaggagacatccacgaaccccacgcagtcgaagaaccactcaccgcttcccctttgtcccaaatgatctctccacgtatcaaaaagcctatcttttcaaacaaatggatgagatacgcatgaagaggaatgtacggttttcttccaagatttgctacattaaaacaggctcttccaccccagacgagaaccctgtacacttctttcatgatttcttctatgaattcaaggtattcatcaagagtcatatcttcatcgtattctttccctacattgtacggaggtgaagtcaccatgagatgtatcgaacagtcaggtatttttttcagaacttctctggcatccccctcaatgaccttgtctaagagattttcaggaagtgggttctcgatgagatcttcctctgaaggttttggaagggaaaagttggaatagatcgctctactataaaatggagatgaatcatggctttcccggccttttactccaaaagaactggttcttgttgattttcttctcttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 22 - DNA, 29 bp, Artificial Sequence
  primer
ggggatccgaatgtggttagtgtgattag

SEQ ID NO: 23 - DNA, 29 bp, Artificial Sequence
  primer
gggaattcttaaccatatcccactagttc